(19) United States
(12) Patent Application Publication
(10) Pub. No.: US 2010/0222393 A1
(43) Pub. Date: Sep. 2, 2010

(54) MUSCARINIC RECEPTOR ANTAGONISTS

(76) Inventors: Naresh Kumar, Gurgaon (IN); Jaskiran Kaur, Amritsar (IN); Venkata P. Palle, Aundh Pune (IN); Beenu Bhatt, Queensland (AU); Shelly Jindal, New Delhi (IN); Anita Chugh, New Delhi (IN); Suman Gupta, Gurgaon (IN); Abhijit Ray, New Delhi (IN); Shivani Malhotra, New Delhi (IN); Raj Kumar Shirumalla, New Delhi (IN)

Correspondence Address: Ranbaxy Inc., Intellectual Property Department, 600 College Road East, PRINCETON, NJ, 08540, US

(21) Appl. No.: 12/159,505

(22) PCT Filed: Dec. 21, 2006
(86) PCT No.: PCT/IB06/55010

§ 371 Date: Mar. 25, 2010

(30) Foreign Application Priority Data
Dec. 30, 2005 (IN) 3521/DEL/2005
Jul. 3, 2006 (IN) 1557/DEL/2006

(51) Int. Cl. A61K 31/4439; C07D 233/54; A61K 31/4164; C07D 401/02; A61P 11/00; A61P 13/00; A61P 1/00; A61P 3/04; A61P 3/10

(52) U.S. Cl. 514/341; 548/335.1; 514/399; 546/272.7

(57) ABSTRACT

Compounds of Formula (I), wherein represents a single bond when G is —OH and double bond when G is —O; R₁ and R₂ are independently selected from hydrogen, alkyl, alkenyl, alkynyl, aralkyl, cycloalkyl, aryl, heteroaryl, heterocyclyl, heterocyclylalkyl or heteroarylalkyl; R₃ is selected from the group selected from hydrogen, hydroxy, alkoxy, alkenyloxy or alkynyloxy; X is selected from oxygen, —NH, —NR (wherein R is alkyl, alkenyl, alkenyl, alkynyl or aryl), sulphur or no atom; Het is heterocyclyl or heteroaryl; n is an integer from 1 to 6; are muscarinic receptor antagonists, which are useful, among other uses, for the treatment of various diseases of the respiratory, urinary and 4 i gastrointestinal systems mediated through muscarinic receptors.

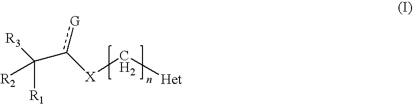

### FIELD OF THE INVENTION

This present invention generally relates to muscarinic receptor antagonists, which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

### BACKGROUND OF THE INVENTION

Physiological effects elicited by the neurotransmitter acetylcholine are mediated through its interaction with two major classes of acetylcholine receptors—the nicotinic and muscarinic acetylcholine receptors. Muscarinic receptors belong to the superfamily of G-protein coupled receptors and five molecularly distinct subtypes are known to exist (M₁, M₂, M₃, M₄ and M₅).

These receptors are widely distributed on multiple organs and tissues and are critical to the maintenance of central and peripheral cholinergic neurotransmission. The regional distribution of these receptor sub-types in the brain and other organs has been documented. (for example, the M₁ subtype is located primarily in neuronal tissues such as cereberal cortex and autonomic ganglia, the M₂ subtype is present mainly in the heart and bladder smooth muscle, and the M₃ subtype is located predominantly on smooth muscle and salivary glands (Nature, 323, p. 411 (1986); Science, 237, p. 527 (1987)).

A review in Curr. Opin. Chem. Biol., 3, p. 426 (1999), as well as in Trends in Pharmacol. Sci., 22, p. 409 (2001) by Eglen et. al., describes the biological potentials of modulating muscarinic receptor subtypes by ligands in different disease conditions, such as Alzheimer's disease, pain, urinary disease condition, chronic obstructive pulmonary disease, and the like.

The pharmacological and medical aspects of the muscarinic class of acetylcholine agonists and antagonists are presented in a review in Molecules, 6, p. 142 (2001). Birdsall et. al. in Trends in Pharmacol. Sci., 22, p. 215 (2001) has also summarized the recent developments on the role of different muscarinic receptor subtypes using different muscarinic receptor of knock out mice.

Almost all the smooth muscles express a mixed population of M₂ and M₃ receptors. Although the M₂-receptors are the predominant cholinoreceptors, the smaller population of M₃-receptors appears to be the most functionally important as they mediate the direct contraction of these smooth muscles. Muscarinic receptor antagonists are known to be useful for treating various medical conditions associated with improper smooth muscle function, such as overactive bladder syndrome, irritable bowel syndrome and chronic obstructive pulmonary disease. However the therapeutic utility of antimuscarinics has been limited by poor tolerability as a result of treatment related, frequent systemic adverse events such as dry mouth, constipation, blurred vision, headache, somnolence and tachycardia. Thus, there exists a need for novel muscarinic receptor antagonists that demonstrate target organ selectivity.

WO 04/005252 discloses azabicyclo derivatives described as musacrinic receptor antagonists. WO 04/004629, WO 04/052857, WO 04/067510, WO 04/014853, WO 04/014363 discloses 3,6-disubstituted azabicyclo [3.1.0]hexane derivatives described as useful muscarinic receptor antagonists. WO 04/056811 discloses flaxavate derivatives as muscarinic receptor antagonists. WO 04/056810 discloses xanthene derivatives as muscarinic receptor antagonists. WO 04/056767 discloses 1-substituted-3-pyrrolidine derivatives as muscarinic receptor antagonists. WO 99/14200, WO 03/1027060, U.S. Pat. No. 6,200,991, and WO 00/56718 disclose heterocycle derivatives as muscarinic receptor antagonists. WO 04/089363, WO 04/089898, WO 04/069835, WO 04/089900 and WO 04/089364 disclose substituted azabicyclohexane derivatives as muscarinic receptor antagonists. WO 06/018708 disclose pyrrolidine derivatives as muscarinic receptor antagonists. WO 06/35303 discloses azabicyclo derivatives as muscarinic receptor antagonists.

J. Med. Chem., 44, p. 984 (2002), describes cyclohexylmethylpiperidinyl-triphenylpropioamide derivatives as selective M₃ antagonist discriminating against the other receptor subtypes. J. Med. Chem., 36, p. 610 (1993), describes the synthesis and antimuscarinic activity of some 1-cycloalkyl-1-hydroxy-1-phenyl-3-(4-substituted piperazinyl)-2-propanones and related compounds. J. Med. Chem., 34, p. 3065 (1991), describes analogues of oxybutynin, synthesis and antimuscarinic activity of some substituted 7-amino-1-hydroxy-5-heptyn-2-ones and related compounds. Bio-Org. Med. Chem. Lett., 15, p. 2093 (2005) describes synthesis and activity of analogues of Oxybutynin and Tolterodine. Chem. Pharm. Bull., 53(4), 437, 2005 discloses thiazole carboxamide derivatives.

The present invention fills the need of muscarinic receptor antagonists useful in the treatment of disease states associated with improper smooth muscle function and respiratory disorders.

### SUMMARY OF THE INVENTION

In one aspect, there are provided muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for the treatment of various diseases of the respiratory, urinary and gastrointestinal systems.

The enantiomers, diastereomers, N-oxides, polymorphs, pharmaceutically acceptable salts and pharmaceutically acceptable solvates of these compounds as well as metabolites having the same type of activity are also provided, as well as pharmaceutical compositions comprising the compounds, their metabolites, enantiomers, diastereomers, N-oxides, polymorphs, solvates or pharmaceutically acceptable salts thereof, in combination with a pharmaceutically acceptable carrier and optionally included excipients.

Other aspects will be set forth in the description which follows, and in part will be apparent from the description or may be learnt by the practice of the invention.

In accordance with one aspect, there are provided compounds having the structure of Formula I:

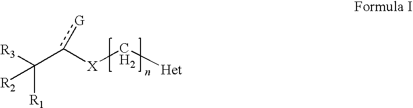

and its pharmaceutically acceptable salts, pharmaceutically acceptable solvates, enantiomers, diastereomers, polymorphs or N-oxides wherein [symbol]
represents a single bond when G is —OH and double bond when G is —O;
R₁ and R₂ are independently selected from hydrogen, alkyl, alkenyl, alkynyl, aralkyl, cycloalkyl, aryl, heteroaryl, heterocyclyl, heterocyclylalkyl or heteroarylalkyl;
R₃ is selected from the group selected from hydrogen, hydroxy, alkoxy, alkenyloxy or alkynyloxy;
X is selected from oxygen, —NH, —NR (wherein R is alkyl, alkenyl, alkenyl, alkynyl or aryl), sulphur or no atom;
Het is heterocyclyl or heteroaryl;
n is an integer from 1 to 6;
With the proviso that when R₁ and R₂ are phenyl, R₃ is hydroxy and X is no atom, then Het cannot be a saturated heterocyclyl group.
The following definitions apply to terms as used herein:

The term “alkyl,” unless otherwise specified, refers to a monoradical branched or unbranched saturated hydrocarbon chain having from 1 to 20 carbon atoms. Alkyl groups can be optionally interrupted by atom(s) or group(s) independently selected from oxygen, sulfur, a phenylene, sulphinyl, sulphonyl group or —NR_{α}—, wherein Rₐ, can be hydrogen, alkyl, cycloalkyl, alkenyl, cycloalkenyl, alkynyl, aryl, acyl, aralkyl, —C(═O)OR_{λ}, SOₘR_{ψ} or —C(═O)NR_{λ}R_{π}. This term can be exemplified by groups such as methyl, ethyl, n-propyl, iso-propyl, n-butyl, iso-butyl, sec-butyl, t-butyl, n-pentyl, isopentyl, neopentyl, n-hexyl, n-decyl, tetradecyl, and the like. Alkyl groups may be substituted further with one or more substituents selected from alkenyl, alkynyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, aryl, heterocyclyl, heteroaryl, (heterocyclyl)alkyl, cycloalkoxy, —CH═N—O(C₁₋₆alkyl), —CH═N—NH(C₁₋₆alkyl), —CH═N—NH(C₁₋₆alkyl)-C₁₋₆alkyl, arylthio, thiol, alkylthio, aryloxy, nitro, aminosulfonyl, aminocarbonylamino, —NHC(═O)R_{λ}, —NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π} {wherein R_{λ} and R_{π} are independently selected from hydrogen, halogen, hydroxy, alkyl, alkenyl, alkynyl, alkenyl, alkoxy, cycloalkyl, cycloalkenyl, aryl, aralkyl, heterocyclyl, heteroaryl, heterocyclylalkyl, heteroarylalkyl or carboxy}, nitro or —SOₘR_{ψ} (wherein m is an integer from 0-2 and R_{ψ} is hydrogen, alkyl, alkenyl, alkynyl, cycloalkyl, aralkyl, aryl, heterocyclyl, heteroaryl, heteroarylalkyl or heterocyclylalkyl). Unless otherwise constrained by the definition, alkyl substituents may be further substituted by 1-3 substituents selected from alkyl, alkenyl, alkynyl, carboxy, —NR_{λ}R_{π}, —C(═O)NR_{λ}R_{π}, —OC(═O)NR_{λ}R_{π}, —HC(═O)NR_{λ}R_{π}, hydroxy, alkoxy, halogen, CF₃, cyano, and —SOₘR_{ψ}; or an alkyl group also may be interrupted by 1-5 atoms of groups independently selected from oxygen, sulfur or —NR_{α}— (wherein R_{α}, R_{λ}, R_{π}, m and R_{ψ} are the same as defined earlier). Unless otherwise constrained by the definition, all substituents may be substituted further by 1-3 substituents selected from alkyl, alkenyl, alkynyl, carboxy, carboxyalkyl, —NR_{λ}R_{π}, —C(═O)NR_{λ}R_{π}, —O—C(═O)NR_{λ}R_{π}, hydroxy, alkoxy, halogen, CF₃, cyano, and —SOₘR_{ψ} (wherein R_{λ}, R_{π}, m and R_{ψ} are the same as defined earlier); or an alkyl group as defined above that has both substituents as defined above and is also interrupted by 1-5 atoms or groups as defined above.

The term “alkenyl,” unless otherwise specified, refers to a monoradical of a branched or unbranched unsaturated hydrocarbon group having from 2 to 20 carbon atoms with cis, trans or geminal geometry. Alkenyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkenyl is attached to a heteroatom, the double bond cannot be alpha to the heteroatom. Alkenyl groups may be substituted further with one or more substituents selected from alkyl, alkenyl, alkynyl, alkoxy, cycloalkyl, cycloalkenyl, acyl, acylamino, acyloxy, —NHC(═O)R_{λ}, —NR_{λ}R_{π}, —C(═O)NR_{λ}R_{π}, —NHC(═O)NR_{λ}R_{π}, —O—C(═O)NR_{λ}R_{π}, alkoxycarbonylamino, azido, cyano, halogen, hydroxy, oxo, keto, carboxyalkyl, thiocarbonyl, carboxy, arylthio, thiol, alkylthio, aryl, aralkyl, aryloxy, heterocyclyl, heteroaryl, heterocyclyl alkyl, heteroaryl alkyl, aminosulfonyl, aminocarbonylamino, alkoxyamino, hydroxyamino, alkoxyamino, nitro or SOₘR_{ψ} (wherein R_{λ}, R_{π}, m and R_{ψ} are as defined earlier). Unless otherwise constrained by the definition, alkenyl substituents optionally may be substituted further by 1-3 substituents selected from alkyl, alkenyl, alkynyl, carboxy, hydroxy, alkoxy, halogen, —CF₃, cyano, —NR_{λ}R_{π}, —C(═O)NR_{λ}R_{π}, —O—C(═O)NR_{λ}R_{π} and —SOₘR_{ψ} (wherein R_{λ}, R_{π}, m and R_{ψ} are as defined earlier). Groups, such as ethenyl or vinyl (CH═CH₂), 1-propylene or allyl (—CH₂CH═CH₂), iso-propylene (—C(CH₃)═CH₂), bicyclo[2.2.1]heptene, and the like, exemplify this term.

The term “alkynyl,” unless otherwise specified, refers to a monoradical of an unsaturated hydrocarbon, having from 2 to 20 carbon atoms. Alkynyl groups can be optionally interrupted by atom(s) or group(s) independently chosen from oxygen, sulfur, phenylene, sulphinyl, sulphonyl and —NR_{α}— (wherein R_{α} is the same as defined earlier). In the event that alkynyl groups are attached to a heteroatom, the triple bond cannot be alpha to the heteroatom. Alkynyl groups may be substituted further with one or more substituents selected from alkyl, alkenyl, alkoxy, cycloalkyl, cycloalkenyl, acyl, acylamino, acyloxy, alkoxycarbonylamino, azido, cyano, halogen, hydroxy, keto, oxo, thiocarbonyl, carboxy, carboxyalkyl, arylthio, thiol, alkylthio, aryl, aralkyl, aryloxy, aminosulfonyl, aminocarbonylamino, hydroxyamino, alkoxyamino, nitro, heterocyclyl, heteroaryl, heterocyclylalkyl, heteroarylalkyl, —NHC(═O)R_{λ}, —NR_{λ}R_{π}, —NHC(═O)NR_{λ}R_{π}, —C(═O)NR_{λ}R_{π}, —O—C(═O)NR_{λ}R_{π} or —SOₘR_{ψ} (wherein R_{λ}, R_{π}, m and R_{ψ} are the same as defined earlier). Unless otherwise constrained by the definition, alkynyl substituents optionally may be substituted further by 1-3 substituents selected from alkyl, alkenyl, alkynyl, carboxy, carboxyalkyl, hydroxy, alkoxy, halogen, CF₃, —NR_{λ}R_{π}, —C(═O)NR_{λ}R_{π}, —NHC(═O)NR_{λ}R_{π}, —C(═O)NR_{λ}R_{π}, cyano or —SOₘR_{ψ} (wherein R_{λ}, R_{π}, m and R_{ψ} are the same as defined earlier).

The term “alkoxy” denotes the group O-alkyl, wherein alkyl is the same as defined above.

The term “aryl,” unless otherwise specified, refers to aromatic system having 6 to 14 carbon atoms, wherein the ring system can be mono-, bi- or tricyclic and are carbocyclic aromatic groups. For example, aryl groups include, but are not limited to, phenyl, biphenyl, anthryl or naphthyl ring and the like, optionally substituted with 1 to 3 substituents selected from halogen (e.g., F, Cl, Br, I), hydroxy, alkyl, alkenyl, alkynyl, cycloalkyl, alkoxy, acyl, aryloxy, CF₃, cyano, nitro, COOR_{ψ}, NHC(═O)R_{λ}, —NR_{λ}R_{π}, —C(═O)NR_{λ}R_{π}, —NHC(═O)NR_{λ}R_{π}, —O—C(═O)NR_{λ}R_{π}, —SOₘR_{ψ}, carboxy, heterocyclyl, heteroaryl, heterocyclylalkyl, heteroarylalkyl or amino carbonyl amino, mercapto, haloalkyl, optionally substituted aryl, optionally substituted heterocyclylalkyl, thioalkyl, —CONHR_{π}, —OCOR_{π}, —COR_{π}, —NHSO₂R_{π} or —SO₂NHR_{π} (wherein R_{λ}, R_{π}, m and R_{ψ} are the same as defined earlier). Aryl groups optionally may be fused with a cycloalkyl group, wherein the cycloalkyl group may optionally contain heteroatoms selected from O, N or S. Groups such as phenyl, naphthyl, anthryl, biphenyl, and the like exemplify this term.

The term “aralkyl,” unless otherwise specified, refers to alkyl-aryl linked through an alkyl portion (wherein alkyl is as defined above) and the alkyl portion contains 1-6 carbon atoms and aryl is as defined below. Examples of aralkyl groups include benzyl, ethylphenyl, propylphenyl, naphthylmethyl and the like.

The term “cycloalkyl,” unless otherwise specified, refers to cyclic alkyl groups of from 3 to 20 carbon atoms having a single cyclic ring or multiple condensed rings, which may optionally contain one or more olefinic bonds, unless otherwise constrained by the definition. Such cycloalkyl groups can include, for example, single ring structures, including cyclopropyl, cyclobutyl, cyclooctyl, cyclopentenyl, and the like or multiple ring structures, including adamantanyl, and bicyclo [2.2.1]heptane or cyclic alkyl groups to which is fused an aryl group, for example, indane, and the like. Spiro and fused ring structures can also be included. Cycloalkyl groups may be substituted further with one or more substituents selected from alkyl, alkenyl, alkynyl, alkoxy, cycloalkyl, cycloalkenyl, acyl, acylamino, aryloxy, alkoxycarbonylamino, azido, cyano, halogen, hydroxy, oxo, thiocarbonyl, carboxy, carboxyalkyl, arylthio, thiol, alkylthio, aryl, aralkyl, aryloxy, aminosulfonyl, aminocarbonylamino, —NR_{λ}R_{π}, —NHC(═O)NR_{λ}R_{π}, —C(═O)NR_{λ}R_{π}, —O—C(═O)NR_{λ}R_{π}, nitro, heterocyclyl, heteroaryl, heterocyclylalkyl, heteroarylalkyl or SOₘR_{ψ} (wherein R_{λ}, R_{π}, m and R_{ψ} are the same as defined earlier). Unless otherwise constrained by the definition, cycloalkyl substituents optionally may be substituted further by 1-3 substituents selected from alkyl, alkenyl, alkynyl, carboxy, hydroxy, alkoxy, halogen, CF₃, —NR_{λ}R_{π}, —C(═O)NR_{λ}R_{π}, —NHC(═O)NR_{λ}R_{π}, —OC(═O)NR_{λ}R_{π}, cyano or —SOₘR_{ψ} (wherein R_{λ}, R_{π}, m and R_{ψ} are the same as defined earlier). “Cycloalkylalkyl” refers to alkyl-cycloalkyl group linked through alkyl portion, wherein the alkyl and cycloalkyl are the same as defined earlier.

The term “carboxy” as defined herein refers to —C(═O)OH.

The term “aryloxy” denotes the group O-aryl, wherein aryl is as defined above.

The term “heteroaryl,” unless otherwise specified, refers to an aromatic ring structure containing 5 or 6 ring atoms or a bicyclic or tricyclic aromatic group having from 8 to 10 ring atoms, with one or more heteroatom(s) independently selected from N, O or S optionally substituted with 1 to 4 substituent(s) selected from halogen (e.g., F, Cl, Br, I), hydroxy, alkyl, alkenyl, alkynyl, cycloalkyl, acyl, carboxy, aryl, alkoxy, aralkyl, cyano, nitro, heterocyclyl, heteroaryl, —NR_{λ}R_{π}, CH═NOH, —(CH₂)_{w}C(═O)R_{η} {wherein w is an integer from 0-4 and R_{η} is hydrogen, hydroxy, OR_{λ}, NR_{λ}R_{π}, —NHOR_{ω} or —NHOH}, —C(═O)NR_{λ}R_{π}—NHC(═O)NR_{λ}R_{π}, —SOₘR_{ψ}, —O—C(═O)NR_{λ}R_{π}, —O—C(═O)R_{λ}, or —O—C(═O)R_{λ} (wherein m, R_{ψ}, R_{λ}, and R_{π} are as defined earlier and R_{ω} is alkyl, cycloalkyl, aryl, heteroaryl, heterocyclyl, heteroarylalkyl or heterocyclylalkyl). Unless otherwise constrained by the definition, the substituents are attached to a ring atom, i.e., carbon or heteroatom in the ring. Examples of heteroaryl groups include oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyridinyl, pyridazinyl, pyrimidinyl, pyrazinyl, thienyl, isoxazolyl, triazinyl, furanyl, benzofuranyl, indolyl, benzthiazinyl, benzthiazinonyl, benzoxazinyl, benzoxazinonyl, quinazonyl, carbazolyl phenothiazinyl, phenoxazinyl, benzothiazolyl or benzoxazolyl, and the like.

The term “heterocyclyl,” unless otherwise specified, refers to a non-aromatic monocyclic or bicyclic cycloalkyl group having 5 to 10 atoms wherein 1 to 4 carbon atoms in a ring are replaced by heteroatoms selected from O, S or N, and optionally are benzofused or fused heteroaryl having 5-6 ring members and/or optionally are substituted, wherein the substituents are selected from halogen (e.g., F, Cl, Br, I), hydroxy, alkyl, alkenyl, alkynyl, cycloalkyl, acyl, optionally substituted aryl, alkoxy, alkaryl, cyano, nitro, oxo, carboxy, optionally substituted heterocyclyl, optionally substituted heterocyclylalkyl, optionally substituted heteroaryl, —O—C(═O)R_{λ}, —O—C(═O)OR_{λ}, —C(═O)NR_{λ}R_{π}, SOₘR_{ψ}, —O—C(═O)NR_{λ}R_{π}, —NHC(═O)NR_{λ}R_{π}, —NR_{λ}R_{π}, mercapto, haloalkyl, thioalkyl, —COOR_{ψ}, —COONHR_{λ}, —COR_{λ}, —NHSO₂R_{λ} or SO₂NHR_{λ} (wherein m, R_{ψ}, R_{λ} and R_{π} are as defined earlier) or guanidine. Heterocyclyl can optionally include rings having one or more double bonds. Such ring systems can be mono-, bi- or tricyclic. Carbonyl or sulfonyl group can replace carbon atom(s) of heterocyclyl. Unless otherwise constrained by the definition, the substituents are attached to the ring atom, i.e., carbon or heteroatom in the ring. Also, unless otherwise constrained by the definition, the heterocyclyl ring optionally may contain one or more olefinic bond(s). Examples of heterocyclyl groups include oxazolidinyl, tetrahydrofuranyl, dihydrofuranyl, benzoxazinyl, benzthiazinyl, imidazolyl, benzimidazolyl, tetrazolyl, carbaxolyl, indolyl, phenoxazinyl, phenothiazinyl, dihydropyridinyl, dihydroisoxazolyl, dihydrobenzofuryl, azabicyclohexyl, thiazolidinyl, dihydroindolyl, pyridinyl, isoindole 1,3-dione, piperidinyl, tetrahydropyranyl, piperazinyl, 3H-imidazo[4,5-b]pyridine, isoquinolinyl, 1H-pyrrolo[2,3-b]pyridine or piperazinyl and the like.

“Heteroarylalkyl” refers to alkyl-heteroaryl group linked through alkyl portion, wherein the alkyl and heteroaryl are as defined earlier.

“Heterocyclylalkyl” refers to alkyl-heterocyclyl group linked through alkyl portion, wherein the alkyl and heterocyclyl are as defined earlier.

“Acyl” refers to —C(═O)R″ wherein R″ is selected from hydrogen, alkyl, cycloalkyl, aryl, aralkyl, heteroaryl, heterocyclyl, heteroarylalkyl or heterocyclylalkyl.

“Thiocarbonyl” refers to —C(═S)H.

“Substituted thiocarbonyl” refers to —C(═S)R″ wherein R″ is selected is the same as defined earlier.

The term “leaving group” refers to groups that exhibit or potentially exhibit the properties of being labile under the synthetic conditions and also, of being readily separated from synthetic products under defined conditions. Examples of leaving groups include, but are not limited to, halogen (e.g., F, Cl, Br, I), triflates, tosylate, mesylates, alkoxy, thioalkoxy, or hydroxy radicals and the like.

The term “protecting groups” refers to moieties that prevent chemical reaction at a location of a molecule intended to be left unaffected during chemical modification of such molecule. Unless otherwise specified, protecting groups may be used on groups, such as hydroxy, amino, or carboxy. Examples of protecting groups are found in T. W. Greene and P. G. M. Wuts, “Protective Groups in Organic Synthesis”, 2^{nd} Ed., John Wiley and Sons, New York, N.Y., which is incorporated herein by reference. The species of the carboxylic protecting groups, amino protecting groups or hydroxy protecting groups employed are not critical, as long as the derivatised moieties/moiety is/are stable to conditions of subsequent reactions and can be removed without disrupting the remainder of the molecule.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like.

The term “pharmaceutically acceptable salts” also refers to salts prepared from pharmaceutically acceptable non-toxic inorganic or organic acid. Examples of such inorganic acids include, but are not limited to, hydrochloric, hydrobromic, hydroiodic, nitrous, nitric, carbonic, sulfuric, phosphoric acid, and the like. Appropriate organic acids include, but are not limited to aliphatic, cycloaliphatic, aromatic, heterocyclic, carboxylic and sulfonic classes of organic acids, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic, methanesulfonic, ethanesulfonic, benzenesulfonic, panthenic, toluenesulfonic, 2-hydroxyethanesulfonic acid and the like.

In accordance with a second aspect, there is provided a method for treatment or prophylaxis of an animal or a human suffering from a disease or disorder of the respiratory, urinary and gastrointestinal systems, wherein the disease or disorder is mediated through muscarinic receptors. The method includes administration of at least one compound having the structure of Formula I.

In accordance with a third aspect, there is provided a method for treatment or prophylaxis of an animal or a human suffering from a disease or disorder associated with muscarinic receptors, comprising administering to a patient in need thereof, an effective amount of a muscarinic receptor antagonist compound as described above.

In accordance with a fourth aspect, there is provided a method for treatment or prophylaxis of an animal or a human suffering from a disease or disorder of the respiratory system such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with a fifth aspect, there are provided processes for preparing the compounds as described above.

The compounds described herein exhibit significant potency in terms of their activity, as determined by in vitro receptor binding and functional assays and in vivo experiments using anaesthetized rabbits. The compounds that were found active in vitro were tested in vivo. Some of the compounds are potent muscarinic receptor antagonists with high affinity towards M₁ and M₃ receptors than M₂ and/or M₅ receptors. Therefore, pharmaceutical compositions for the possible treatment for the disease or disorders associated with muscarinic receptors are provided. In addition, the compounds can be administered orally or parenterally.

### DETAILED DESCRIPTION OF THE INVENTION

The compounds disclosed herein may be prepared by methods represented by the reaction sequences, for example, as generally shown in Scheme I

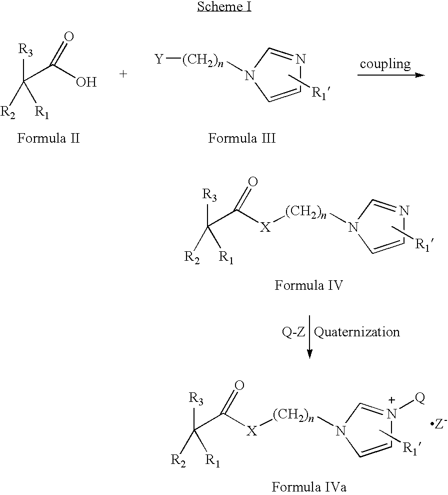

The compounds of Formula IV can be prepared, for example, by following the procedure as depicted in, for example, Scheme I wherein the reaction comprises reacting a compound of Formula II (wherein R₁, R₂ and R₃ are the same as defined earlier) with a compound of Formula III [wherein n is an integer from 1-6 and Y is —OH, —Omesyl, —Otosyl, —Otriflyl or —NH₂.HCl or —NHR. HCl wherein R is the same as defined earlier and R₁′ is selected from hydrogen, alkyl, alkenyl, alkynyl, aralkyl, cycloalkyl, aryl, heteroaryl, heterocyclyl, heterocyclylalkyl or heteroarylalkyl and is always a substitutent on the carbon atoms of imidazolyl ring) to give a compound of Formula IV (wherein X is the same as defined earlier). The compound of Formula IV can be further quaternized with a compound of Formula Q-Z (wherein Q can be selected from alkyl, alkenyl, alkynyl, cycloalkyl, heterocyclyl, aryl, heteroaryl, aralkyl, heteroarylalkyl or heterocyclylalkyl and Z is an anion disclosed in Int. J. Pharmaceutics, 33 (1986), page 202, for example, but not limited to, acetate, tartarate, chloride, bromide, iodide, sulphate, phosphate, nitrate, carbonate, fumarate, glutamate, citrate, methanesulphonate, toulenesulphonate, benzenesulphonate, maleate or succinate) to give a compound of Formula IVa.

The coupling of a compound of Formula II with a compound of Formula III (when Y is —NH.HCl or —NHR.HCl) can be carried out in an organic solvent (for example, dimethylformamide, chloroform, tetrahydrofuran, diethyl ether or dioxane) in the presence of a base (for example, N-methylmorpholine, triethylamine, diisopropylethylamine or pyridine) with a condensing agent (for example, 1-(3-dimethylaminopropyl)-3-ethylcarbodiimide hydrochloride (EDC.HCl) or dicyclohexylcarbodiimide (DCC)).

The coupling of a compound of Formula II with a compound of Formula III (when Y is —OH or —SH) can be carried out in an organic solvent, for example, tetrahydrofuran, dimethylformamide, diethyl ether or dioxane in the presence of a coupling agent, for example, carbonyldiimidazole 1-(3-dimethylaminopropyl)-3-ethylcarbodiimide hydrochloride (EDC.HCl) or dicyclohexylcarbodiimide (DCC).

Alternatively, coupling of a compound of Formula II with a compound of Formula III (when Y is —OH or —SH) can be carried out in an organic solvent (for example, toluene, heptane or xylene) in the presence of a base (for example, sodium hydride or sodium methoxide) to give a compound of Formula IV.

The coupling of a compound of Formula II with a compound of Formula III (when Y is —Omesyl, —Otosyl or —Otriflyl) can be carried out in an organic solvent (for example, toluene, heptane or xylene) in the presence of a base (for example, 1,8-diazabicyclo[5.4.0]undecen-7-ene (DBU) or 1,4-diazabicyclo[2.2.2]octane) to give a compound of Formula IV.

The quaternization of a compound of Formula IV to give a compound of Formula IVa can be carried out by reacting the compound of Formula IV with a compound of Formula Q-Z in an optional organic solvent such as, for example acetonitrile, dichloromethane, dichloroethane, carbon tetrachloride, chloroform, toluene, benzene, DMF, DMSO. Particular illustrative compounds which may be prepared by, for example Scheme I include:
2-Cyclopentyl-2-hydroxy-N-[2-(1H-imidazol-1-yl)ethyl]-2-phenylacetamide (Compound No. 1)
1H-Imidazol-1-ylmethyl cyclohexyl(hydroxy)(4-methylphenyl)acetate (Compound No. 2)
2-(4-Fluorophenyl)-2-hydroxy-N-[2-(1H-imidazol-1-yl)ethyl]-2-phenylacetamide (Compound No. 3)
2-Cyclobutyl-2-hydroxy-N-[2-(1H-imidazol-1-yl)ethyl]-2-phenylacetamide (Compound No. 4)
2-Cyclopentyl-2-(4-fluorophenyl)-2-hydroxy-N-[2-(1H-imidazol-1-yl)ethyl]acetamide (Compound No. 5)
2-Hydroxy-N-[2-(1H-imidazol-1-yl)ethyl]-2,2-diphenylacetamide (Compound No. 6)
2-Cyclohexyl-2-hydroxy-N-[2-(1H-imidazol-1-yl)ethyl]-2-phenylacetamide (Compound No. 7)
2-Cyclopentyl-2-hydroxy-N-[2-(2-isopropyl-1H-imidazol-1-yl)ethyl]-2-phenylacetamide (Compound No. 8)
2-Cyclohexyl-2-hydroxy-N-[2-(2-isopropyl-1H-imidazol-1-yl)ethyl]-2-phenylacetamide (Compound No. 9)
2-Hydroxy-N-[2-(2-isopropyl-1H-imidazol-1-yl)ethyl]-2-phenyl-2-pyridin-3-ylacetamide (Compound No. 10)
2-(4-Fluorophenyl)-2-hydroxy-N-[2-(2-isopropyl-1H-imidazol-1-yl)ethyl]-2-phenylacetamide (Compound No. 11)
2-Hydroxy-N-[2-(2-isopropyl-1H-imidazol-1-yl)ethyl]-2,2-diphenylacetamide (Compound No. 12)
2-(2-Methyl-1H-imidazol-1-yl)ethyl cyclopentyl(hydroxy)phenylacetate (Compound No. 13)
2-(2-Methyl-1H-imidazol-1-yl)ethyl (2R)-cyclopentyl(hydroxy)phenylacetate (Compound No. 14)
(2R)-2-cyclopentyl-2-hydroxy-N-[2-(2-methyl-1H-imidazol-1-yl)ethyl]-2-phenylacetamide (Compound No. 16)
2-Hydroxy-N-[2-(1H-imidazol-1-yl)ethyl]-2-phenyl-2-pyridin-3-ylacetamide (Compound No. 17)
2-Cyclopentyl-2-hydroxy-N-[2-(2-methyl-1H-imidazol-1-yl)ethyl]-2-phenylacetamide (Compound No. 18)
2-Hydroxy-N-[2-(2-methyl-1H-imidazol-1-yl)ethyl]-2-phenyl-2-pyridin-3-ylacetamide (Compound No. 19)
2-Cyclohexyl-2-hydroxy-N-[2-(2-methyl-1H-imidazol-1-yl)ethyl]-2-phenylacetamide (Compound No. 20)
2-(4-Fluorophenyl)-2-hydroxy-N-[2-(2-methyl-1H-imidazol-1-yl)ethyl]-2-phenylacetamide (Compound No. 21)
2-Cyclopentyl-2-(4-fluorophenyl)-2-hydroxy-N-[2-(2-methyl-1H-imidazol-1-yl)ethyl]acetamide (Compound No. 22)
2-Cyclobutyl-2-hydroxy-N-[2-(2-methyl-1H-imidazol-1-yl)ethyl]-2-phenylacetamide (Compound No. 23)
2-Hydroxy-N-[2-(2-methyl-1H-imidazol-1-yl)ethyl]-2,2-diphenylacetamide (Compound No. 24)
3,3,3-Trifluoro-2-hydroxy-N-[2-(2-methyl-1H-imidazol-1-yl)ethyl]-2-(4-methylphenyl)propanamide (Compound No. 25)
N-[2-(2-methyl-1H-imidazol-1-yl)ethyl]-2,2-diphenylacetamide (Compound No. 26)
-Cyclopentyl-N-[2-(2-methyl-1H-imidazol-1-yl)ethyl]-2-phenylacetamide (Compound No. 27)
2-Cyclopentyl-2-hydroxy-N-[2-(2-methyl-1H-imidazol-1-yl)ethyl]-2-(4-methylphenyl)acetamide (Compound No. 28)
2-Cyclohexyl-2-hydroxy-N-[2-(2-methyl-1H-imidazol-1-yl)ethyl]-2-(4-methylphenyl)acetamide (Compound No. 29)
2-Hydroxy-N-[2-(2-methyl-1H-imidazol-1-yl)ethyl]-2-(4-methylphenyl)-2-phenylacetamide (Compound No. 30)
2-Cyclopentyl-2-hydroxy-N-[3-(1H-imidazol-1-yl)propyl]-2-phenylacetamide (Compound No. 31)
2-Cyclohexyl-2-hydroxy-N-[3-(1H-imidazol-1-yl)propyl]-2-phenylacetamide (Compound No. 32)
2-Cyclopentyl-2-hydroxy-N-[3-(2-methyl-1H-imidazol-1-yl)propyl]-2-phenylacetamide (Compound No. 33)
2-Cyclohexyl-2-hydroxy-N-[3-(2-methyl-1H-imidazol-1-yl)propyl]-2-phenylacetamide (Compound No. 34)
(2R)-2-(3,3-Difluorocyclopentyl)-2-hydroxy-N-[3-(2-methyl-1H-imidazol-1-yl)propyl]-2-phenylacetamide (Compound No. 35)
2-Cyclopentyl-2-hydroxy-N-methyl-N-[3-(2-methyl-1H-imidazol-1-yl)propyl]-2-phenylacetamide (Compound No. 36)
2-Cyclohexyl-2-hydroxy-N-methyl-N-[3-(2-methyl-1H-imidazol-1-yl)propyl]-2-phenylacetamide (Compound No. 37)
2-Cyclopentyl-2-hydroxy-N-[2-(1H-imidazol-1-yl)ethyl]-N-methyl-2-phenylacetamide (Compound No. 38)
1H-imidazol-1-ylmethyl cyclopentyl(hydroxy)phenylacetate (Compound No. 46)
1H-imidazol-1-ylmethyl cyclohexyl(hydroxy)phenylacetate (Compound No. 47)
1H-imidazol-1-ylmethyl (2R)-cyclopentyl(hydroxy)phenylacetate (Compound No. 48)
1H-Imidazol-1-ylmethyl cyclopentyl(hydroxy)(4-methoxyphenyl)acetate (Compound No. 49)
2-Cyclohexyl-2-hydroxy-N-[2-(1H-imidazol-1-yl)ethyl]-N-methyl-2-phenylacetamide (Compound No. 57)
2-Cyclopentyl-2-hydroxy-N-[2-(1H-imidazol-1-yl)ethyl]-N-methyl-2-phenylacetamide (Compound No. 58)
2-(2-Methyl-1H-imidazol-1-yl)ethyl cyclopentyl(phenyl)acetate (Compound No. 59)
2-(2-Methyl-1H-imidazol-1-yl)ethyl cyclohexyl(hydroxy)phenylacetate (Compound No. 60)
3-(2-Methyl-1H-imidazol-1-yl)propyl cyclopentyl(hydroxy)phenylacetate (Compound No. 61)
3-(2-Methyl-1H-imidazol-1-yl)propyl (2R)-[(1R)-3,3-difluorocyclopentyl](hydroxy)phenylacetate (Compound No. 62)
3-(2-Methyl-1H-imidazol-1-yl)propyl 2R-2-(1S or 1R)(3,3-difluorocyclohexyl) (hydroxy)phenylacetate (Compound No. 63)
2-(2-Isopropyl-1H-imidazol-1-yl)ethyl cyclohexyl(hydroxy)phenylacetate (Compound No. 64)
2-(1H-Imidazol-1-yl)ethyl cyclopentyl(hydroxy)phenylacetate (Compound No. 65)
2-(2-Methyl-1H-imidazol-1-yl)ethyl (2R)-[(1S)-3,3-difluorocyclopentyl](hydroxy)phenylacetate (Compound No. 66)
2-(1H-Imidazol-1-yl)ethyl cyclohexyl(hydroxy)phenylacetate (Compound No. 67)
2-(2-Isopropyl-1H-imidazol-1-yl)ethyl cyclopentyl(hydroxy)phenylacetate (Compound No. 68)
2-(2-Methyl-1H-imidazol-1-yl)ethyl (2R)-[(1R)-3,3-difluorocyclopentyl](hydroxy)phenylacetate (Compound No. 69)
2-(2-Isopropyl-1H-imidazol-1-yl)ethyl cycloheptyl(hydroxy)phenylacetate (Compound No. 70)
2-(2-Methyl-1H-imidazol-1-yl)ethyl cycloheptyl(hydroxy)phenylacetate (Compound No. 71)
3-Benzyl-1-(2-{[cycloheptyl(hydroxy)phenylacetyl]oxy}ethyl)-2-isopropyl-1H-imidazol-3-ium bromide (Compound No. 72)
3-Benzyl-1-[2-({(2R)-2-[(1S)-3,3-difluorocyclopentyl]-2-hydroxy-2-phenylacetyl}oxy)ethyl]-2-isopropyl-1H-imidazol-3-ium bromide (Compound No. 73)
3-Benzyl-1-[2-({(2R)-2-[(1R)-3,3-difluorocyclopentyl]-2-hydroxy-2-phenylacetyl}oxy)ethyl]-2-isopropyl-1H-imidazol-3-ium bromide (Compound No. 74)
3-(4-Bromobenzyl)-1-[2-({(2R)-2-[(1R)-3,3-difluorocyclopentyl]-2-hydroxy-2-phenylacetyl}oxy)ethyl]-2-isopropyl-1H-imidazol-3-ium bromide (Compound No. 75)
3-Benzyl-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 76)
3-(4-Bromobenzyl)-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 77)
3-(4-Fluorobenzyl-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 78)
3-Benzyl-1-(2-{[cycloheptyl(hydroxy)phenylacetyl]oxy}ethyl)-2-methyl-1H-imidazol-3-ium bromide (Compound No. 79).
3-(4-Bromobenzyl)-1-(2-{[cycloheptyl(hydroxy)phenylacetyl]oxy}ethyl)-2-methyl-1H-imidazol-3-ium bromide (Compound No. 80)
1-[2-({(2R)-2-[(1S)-3,3-difluorocyclopentyl]-2-hydroxy-2-phenylacetyl}oxy)ethyl]-2-isopropyl-3-methyl-1H-imidazol-3-ium iodide (Compound No. 81)
1-[2-({(2R)-2-[(1R)-3,3-difluorocyclopentyl]-2-hydroxy-2-phenylacetyl}oxy)ethyl]-3-(4-fluorobenzyl)-2-isopropyl-1H-imidazol-3-ium bromide (Compound No. 82)
1-(2-{[2-Cyclohexyl-2-hydroxy-2-phenylacetyl]oxy}ethyl)-2-isopropyl-3-methyl-1H-imidazol-3-ium iodide (Compound No. 83)
1-(2-{[Cyclopentyl (hydroxy)phenylacetyl]oxy}ethyl)-3-methyl-1H-imidazol-3-ium iodide (Compound No. 84)
1-[2-({(2R)-2-[(1S)-3,3-difluorocyclopentyl]-2-hydroxy-2-phenylacetyl}oxy)ethyl]-2,3-dimethyl-1H-imidazol-3-ium iodide (Compound No. 85)
1-[2-({(2R)-2-[(1R)-3,3-difluorocyclopentyl]-2-hydroxy-2-phenylacetyl}oxy)ethyl]-2,3-dimethyl-1H-imidazol-3-ium iodide (Compound No. 86)
1-(2-{[Cyclohexyl(hydroxy)phenylacetyl]oxy}ethyl)-3-methyl-1H-imidazol-3-ium iodide (Compound No. 87)
1-(2-{[Cyclopentyl(hydroxy)phenylacetyl]oxy}ethyl)-2-isopropyl-3-methyl-1H-imidazol-3-ium iodide (Compound No. 88)
1-(2-{[Cyclopentyl(hydroxy)phenylacetyl]amino}ethyl)-2,3-dimethyl-1H-imidazol-3-ium iodide (Compound No. 89)
1-(2-{[Cyclohexyl(hydroxy)phenylacetyl]amino}ethyl)-2-isopropyl-3-methyl-1H-imidazol-3-ium iodide (Compound No. 90)
1-(2-{[Cycloheptyl(hydroxy)phenylacetyl]oxy}ethyl)-2,3-dimethyl-1H-imidazol-3-ium iodide (Compound No. 91)
1-(2-{[Cycloheptyl(hydroxy)phenylacetyl]oxy}ethyl)-2-isopropyl-3-methyl-1H-imidazol-3-ium iodide (Compound No. 92.)
1-[2-({(2R)-2-[(1R)-3,3-difluorocyclopentyl]-2-hydroxy-2-phenylacetyl}oxy)ethyl]-2-isopropyl-3-methyl-1H-imidazol-3-ium iodide (Compound No. 93)
1-(3-{[(2R)-2-(3,3-difluorocyclopentyl)-2-hydroxy-2-phenylacetyl]amino}propyl)-2,3-dimethyl-1H-imidazol-3-ium iodide (Compound No. 94)
1-(3-{[Cyclopentyl(hydroxy)phenylacetyl]amino}propyl)-2,3-dimethyl-1H-imidazol-3-ium iodide (Compound No. 95)
1-(3-{[Cyclohexyl(hydroxy)phenylacetyl]amino}propyl)-2,3-dimethyl-1H-imidazol-3-ium iodide (Compound No. 96)
1-(2-{[Cyclopentyl(hydroxy)phenylacetyl](methyl)amino}ethyl)-3-methyl-1H-imidazol-3-ium iodide (Compound No. 97)
1-(3-{[Cyclopentyl(hydroxy)phenylacetyl](methyl)amino}propyl)-2,3-dimethyl-1H-imidazol-3-ium iodide (Compound No. 98)
1-(3-{[Cyclopentyl(hydroxy)phenylacetyl]oxy}propyl)-2,3-dimethyl-1H-imidazol-3-ium iodide (Compound No. 99)
1-[3-({(2R)-2-[(1S)-3,3-Difluorocyclopentyl]-2-hydroxy-2-phenylacetyl}oxy)propyl]-2,3-dimethyl-1H-imidazol-3-ium iodide (Compound No. 100)
3-Benzyl-1-[3-({(2R)-2-[(1S)-3,3-difluorocyclopentyl]-2-hydroxy-2-phenylacetyl}oxy)propyl]-2-methyl-1H-imidazol-3-ium bromide (Compound No. 101)
3-(4-Bromobenzyl)-1-[3-({(2R)-2-[(1S)-3,3-difluorocyclopentyl]-2-hydroxy-2-phenylacetyl}oxy)propyl]-2-methyl-1H-imidazol-3-ium bromide (Compound No. 102)
1-[3-({(2R)-2-[(1R)-3,3-difluorocyclopentyl]-2-hydroxy-2-phenylacetyl}oxy)propyl]-2,3-dimethyl-1H-imidazol-3-ium iodide (Compound No. 103)
3-Benzyl-1-[3-({(2R)-2-[(1R)-3,3-difluorocyclopentyl]-2-hydroxy-2-phenylacetyl}oxy)propyl]-2-methyl-1H-imidazol-3-ium bromide (Compound No. 104).
3-(4-Bromobenzyl)-1-[3-({(2R)-2-[(1R)-3,3-difluorocyclopentyl]-2-hydroxy-2-phenylacetyl}oxy)propyl]-2-methyl-1H-imidazol-3-ium bromide (Compound No. 105)
1-[3-({(2R)-2-[(1S or 1R)-3,3-difluorocyclohexyl]-2-hydroxy-2-phenylacetyl}oxy)propyl]-2,3-dimethyl-1H-imidazol-3-ium bromide (Compound No. 106)
3-Benzyl-1-[3-({(2R)-2-[(1R or 1S)-3,3-difluorocyclohexyl]-2-hydroxy-2-phenylacetyl}oxy)propyl]-2-methyl-1H-imidazol-3-ium bromide (Compound No. 107)
3-(4-Bromobenzyl)-1-[3-({(2R)-2-[(1R or 1S)-3,3-difluorocyclohexyl]-2-hydroxy-2-phenylacetyl}oxy)propyl]-2-methyl-1H-imidazol-3-ium bromide (Compound No. 108)
1-(2-{[Cyclopentyl(hydroxy)phenylacetyl](methyl)amino}ethyl)-2,3-dimethyl-1H-imidazol-3-ium iodide (Compound No. 109)
3-(2-Methyl-1H-imidazol-1-yl)propyl (2R)-[(1S)-3,3-difluorocyclopentyl](hydroxy)phenylacetate (Compound No. 110)
1-(2-{[Cyclohexyl(hydroxy)phenyl acetyl]oxy}ethyl)-2,3-dimethyl-1H-imidazol-3-ium iodide (Compound No 111)
2-(2-Isopropyl-1H-imidazol-1-yl)ethyl (2R)-[(1S)-3,3-difluorocyclopentyl](hydroxy)phenylacetate (Compound No. 112), and
2-(2-Isopropyl-1H-imidazol-1-yl)ethyl (2R)-[(1R)-3,3-difluorocyclopentyl](hydroxy)phenylacetate (Compound No. 113),

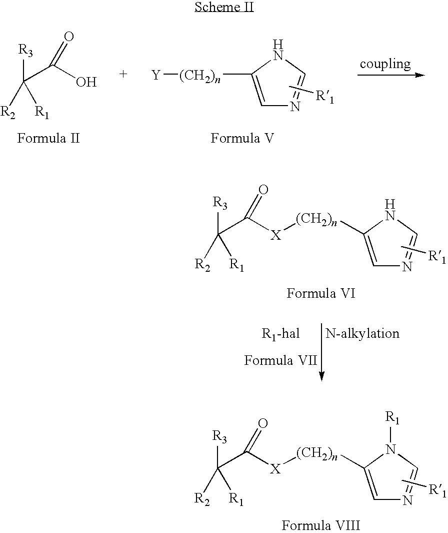

The compounds of Formula VIII can be prepared, for example, by following the procedure as described in, for example, Scheme II wherein the reaction comprises reacting a compound of Formula II (wherein R₁, R₂ and R₃ are the same as defined earlier) with a compound of Formula V (wherein Y, n and R₁′ are the same as defined earlier) to give a compound of Formula VI (wherein X is the same as defined earlier) which is reacted with a compound of Formula VII (wherein R₁ is the same as defined earlier and hal is Br, Cl or I) to give a compound of Formula VIII.

The coupling of a compound of Formula II with a compound of Formula V (when Y is —NH.HCl, —NHR.HCl) to give a compound of Formula VI can be carried out in an organic solvent (for example, dimethylformamide, chloroform, tetrahydrofuran, diethyl ether or dioxane) in the presence of a base (for example, N-methylmorpholine, triethylamine, diisopropylethylamine or pyridine) with a condensing agent (for example, 1-(3-dimethylaminopropyl)-3-ethylcarbodiimide hydrochloride (EDC.HCl) or dicyclohexylcarbodiimide (DCC)).

The coupling of a compound of Formula II with a compound of Formula V (when Y is —OH or —SH) to give a compound of Formula VI can be carried out in an organic solvent, for example, dimethylformamide, tetrahydrofuran in the presence of carbonyldiimidazole and an optional base such as sodium hydride, triethylamine, N-ethyldiisopropylamine or pyridine.

Alternatively, coupling of a compound of Formula II with a compound of Formula V (when Y is —OH or —SH) can be carried out in an organic solvent (for example, toluene, heptane or xylene) in the presence of a base (for example, sodium hydride or sodium methoxide).

The coupling of a compound of Formula II with a compound of Formula V (when Y is —Omesyl, —Otosyl or —Otriflyl) to give a compound of Formula VI can be carried out in an organic solvent (for example, toluene, heptane or xylene) in the presence of a base (for example, 1,8-diazabicyclo[5.4.0]undecen-7-ene (DBU) or 1,4-diazabicyclo[2.2.2]octane).

The N-derivatization of a compound of Formula VI with a compound of Formula VII to give a compound of Formula VIII can be carried out in an organic solvent (for example, acetonitrile, dichloromethane, chloroform or carbon tetrachloride) in the presence of a base (for example, potassium carbonate, sodium carbonate or sodium bicarbonate).

Particular illustrative compounds which may be prepared, for example, by following Scheme II made:
2-Cyclopentyl-2-hydroxy-N-[2-(1H-imidazol-4-yl)ethyl]-2-phenylacetamide (Compound No. 39)
N-[2-(1-benzyl-1H-imidazol-4-yl)ethyl]-2-cyclopentyl-2-hydroxy-2-(4-methylphenyl)acetamide (Compound No. 40)
2-Cyclopentyl-2-hydroxy-N-[2-(1H-imidazol-4-yl)ethyl]-2-(4-methylphenyl)acetamide (Compound No. 41)
2-Cyclohexyl-2-hydroxy-N-[2-(1H-imidazol-4-yl)ethyl]-2-(4-methylphenyl)acetamide (Compound No. 42)
2-Hydroxy-N-[2-(1H-imidazol-4-yl)ethyl]-2,2-diphenylacetamide (Compound No. 43)
N-[2-(1-benzyl-1H-imidazol-4-yl)ethyl]-2-cyclohexyl-2-hydroxy-2-(4-methylphenyl)acetamide (Compound No. 44), and
N-[2-(1-benzyl-1H-imidazol-4-yl)ethyl]-2-hydroxy-2,2-diphenylacetamide (Compound No. 45).

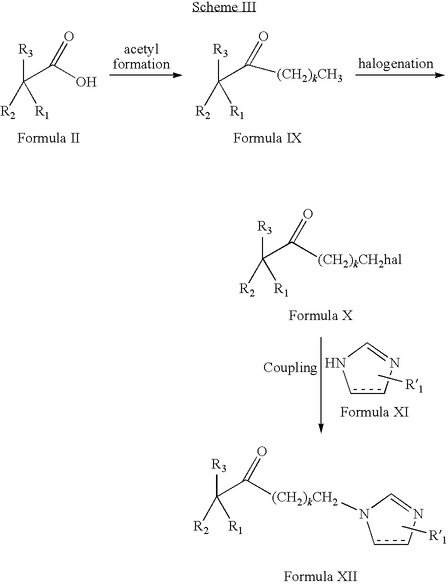

The compounds of Formula XII can be prepared, for example, by following the procedure depicted in, for example, Scheme III wherein the compound of Formula II (wherein R₁, R₂ and R₃ are the same as defined earlier) undergoes acylation to give a compound of Formula IX (wherein k is an integer from 0-3), which undergoes halogenation to give a compound of Formula X (wherein hal is the same as defined earlier), which undergoes coupling with a compound of Formula XI (wherein [symbol] represents single bond or double bond and R₁ is the same as defined earlier) to give a compound of Formula XII.

The acylation of the compound of Formula II to give a compound of Formula IX can be carried out with alkyl lithium in an organic solvent (for example, tetrahydrofuran, dimethylformamide, dioxane or diethylether) in the presence of an optional base (for example, butyl lithium, N-methylmorpholine, pyridine or triethylamine).

The halogenation of a compound of Formula IX to give a compound of Formula X can be carried out with halogenating agent (for example, pyridinium tribromide, phosphorous pentachloride, phosphorous tribromide, phosphorous pentachloride or thionyl chloride) in an organic solvent (for example, tetrahydrofuran, dimethylformamide, diethylether or dioxane).

The coupling of a compound of Formula X with a compound of Formula XI to give a compound of Formula XII can be carried out in the presence of a base (for example, triethylamine, pyridine, N-methylmorpholine or diisopropylethylamine) in an organic solvent for example, dichloromethane, dichloroethane, carbon tetrachloride or chloroform.

Particular illustrative compounds include these shown below:
1-Cyclopentyl-1-hydroxy-1-(4-methoxyphenyl)-3-(2-methyl-1H-imidazol-1-yl)acetone (Compound No. 15)
1-Cyclohexyl-1-hydroxy-3-(1H-imidazol-1-yl)-1-phenylacetone (Compound No. 50)
1-Cyclohexyl-1-hydroxy-3-(2-methyl-1H-imidazol-1-yl)-1-phenylacetone (Compound No. 51)
1-Cyclopentyl-1-hydroxy-3-(2-isopropyl-1H-imidazol-1-yl)-1-(4-methoxyphenyl)acetone (Compound No. 52)
1-Cyclohexyl-1-hydroxy-3-(2-isopropyl-1H-imidazol-1-yl)-1-phenylacetone (Compound No. 53)
1-Cyclohexyl-1-hydroxy-3-(2-methyl-4,5-dihydro-1H-imidazol-1-yl)-1-phenylacetone (Compound No. 54)
1-Cyclopentyl-1-hydroxy-1-(4-methoxyphenyl)-3-(2-methyl-4,5-dihydro-1H-imidazol-1-yl)acetone (Compound No. 55), and
1-Cyclopentyl-1-hydroxy-3-(1H-imidazol-1-yl)-1-(4-methoxyphenyl)acetone (Compound No. 56),

In the above schemes, where specific bases, condensing agents, protecting groups, deprotecting agents, solvents, catalysts, temperatures, etc. are mentioned, it is to be understood that other bases, condensing agents, protecting groups, deprotecting agents, solvents, catalysts, temperatures, etc. known to those skilled in the art may be used. Similarly, the reaction temperature and duration may be adjusted according to the desired needs.

Suitable salts of the compounds represented by the Formula I were prepared so as to solubilize the compound in aqueous medium for biological evaluations, as well as to be compatible with various dosage formulations and also to aid in the bioavailability of the compounds. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, tolounesulphonate and methanesulphonate). When carboxyl groups are included in the Formula I as substituents, they may be present in the form of an alkaline or alkali metal salt (for example, sodium, potassium, calcium, magnesium, and the like). These salts may be prepared by various techniques, such as treating the compound with an equivalent amount of inorganic or organic, acid or base in a suitable solvent.

The compounds described herein can be produced and formulated as their enantiomers, diastereomers, N-Oxides, polymorphs, solvates and pharmaceutically acceptable salts, as well as metabolites having the same type of activity. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

Where desired, the compounds of Formula I and/or their pharmaceutically acceptable salts, pharmaceutically acceptable solvates, stereoisomers, tautomers, racemates, prodrugs, metabolites, polymorphs or N-oxides may be advantageously used in combination with one or more other therapeutic agents. Examples of other therapeutic agents, which may be used in combination with compounds of Formula I of this invention and/or their pharmaceutically acceptable salts, pharmaceutically acceptable solvates, stereoisomers, tautomers, racemates, prodrugs, metabolites, polymorphs or N-oxides include but are not limited to, corticosteroids, beta agonists, leukotriene antagonists, 5-lipoxygenase inhibitors, anti-histamines, antitussives, dopamine receptor antagonists, chemokine inhibitors, p38 MAP Kinase inhibitors, and PDE-IV inhibitors.

The compositions can be administered by inhalation. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients. The compositions can be administered by the nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered nasally from devices, which deliver the formulation in an appropriate manner.

Alternatively, compositions can be administered orally, rectally, parenterally (intravenously, intramuscularly or subcutaneously), intracisternally, intravaginally, intraperitoneally or topically.

Solid dosage forms for oral administration may be presented in discrete units, for example, capsules, cachets, lozenges, tablets, pills, powders, dragees or granules, each containing a predetermined amount of the active compound. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms can be prepared with coatings and shells, such as enteric coatings and others well known in this art. They may contain opacifying agents, and can also be of such composition that they release the active compound or compounds in a certain part of the intestinal tract in a delayed manner. Examples of embedding compositions which can be used are polymeric substances and waxes.

The active compounds can also be in micro-encapsulated form, if appropriate, with one or more of the above mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, for example, wetting agents, emulsifying and suspending agents, sweetening, flavoring and perfuming agents, colorants or dyes.

Suspensions, in addition to the active compounds, may contain suspending agents, as for example, ethoxylated isostearyl alcohols, polyoxyethylene sorbitol and sorbitan esters, microcrystalline cellulose, aluminium metahydroxide, bentonite, agar-agar and tragacanth, or mixtures of these substances, and the like.

Dosage forms for topical administration of a compound of this invention include powder, spray, inhalant, ointment, creams, salve, jelly, lotion, paste, gel, aerosol, or oil. The active component is admixed under sterile conditions with a pharmaceutically acceptable carrier and any needed preservatives, buffers or propellants as may be required. Opthalmic formulations, eye ointments, powders and solutions are also contemplated as being within the scope of this invention.

Compositions suitable for parenteral injection may comprise pharmaceutically acceptable sterile aqueous or nonaqueous solutions, dispersions, suspensions or emulsions and sterile powders for reconstitution into sterile injectable solutions or dispersions. These preparations may contain anti-oxidants, buffers, bacteriostats and solutes, which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monosterate and gelatin.

Suppositories for rectal administration of the compound of Formula I can be prepared by mixing the drug with a suitable nonirritating excipient such as cocoa butter and polyethylene glycols or a suppository wax, which are solid at ordinary temperatures but liquid at body temperature and which therefore melt in the rectum or vaginal cavity and release the drug.

If desired, and for more effective distribution, the compounds can be incorporated into slow release or targeted delivery systems such as polymer matrices, liposomes, and microspheres. They may be sterilized, for example, by filtration through a bacteria-retaining filter, or by incorporating sterilizing agents in the form of sterile solid compositions which can be dissolved in sterile water, or some other sterile injectable medium immediately before use.

Actual dosage levels of active ingredient in the compositions of the invention and spacing of individual dosages may be varied so as to obtain an amount of active ingredient that is effective to obtain a desired therapeutic response for a particular composition and method of administration. It will be understood, however, that the specific dose level for any particular patient will depend upon a variety of factors including the compound chosen, the body weight, general health, sex, diet, route of administration, the desired duration of treatment, rates of absorption and excretion, combination with other drugs and the severity of the particular disease being treated and is ultimately at the discretion of the physician.

The pharmaceutical compositions described herein can be produced and administered in dosage units, each unit containing a certain amount of at least one compound described herein and/or at least one physiologically acceptable addition salt thereof. The dosage may be varied over extremely wide limits, as the compounds are effective at low dosage levels and relatively free of toxicity. The compounds may be administered in the low micromolar concentration, which is therapeutically effective, and the dosage may be increased as desired up to the maximum dosage tolerated by the patient.

The examples mentioned below demonstrate general synthetic procedures, as well as specific preparations of particular compounds. The examples are provided to illustrate the details of the invention and should not be constrained to limit the scope of the present invention.

### EXAMPLES

Various solvents, such as acetone, methanol, pyridine, ether, tetrahydrofuran, hexanes, and dichloromethane, were dried using various drying reagents according to procedures described in the literature. IR spectra were recorded as nujol mulls or a thin neat film on a Perkin Elmer Paragon instrument, Nuclear Magnetic Resonance (NMR) were recorded on a Varian XL-300 MHz or Bruker 400 MHz instrument using tetramethylsilane as an internal standard.

### Synthesis of tert-butyl [2-(1H-imidazol-1-yl)ethyl]carbamate

### Step a: Synthesis of tert-butyl (2-bromoethyl)carbamate

To a solution of hydrobromide salt of 2-bromoethylamine (20 g, 97 mmol) in dichloromethane (250 ml) was added triethyl amine (34.01 ml, 244 mmol) followed by the addition of tert-butoxycarbonyl anhydride (24.63 ml, 107 mmol) at 0-5° C. and the reaction mixture was stirred at room temperature overnight. The reaction mixture was washed with saturated sodium bicarbonate solution. The mixture was extracted with ethyl acetate and the organic layer was washed with water and brine, dried over anhydrous sodium sulphate and concentrated under reduced pressure to furnish the title compound. Yield: 20 g.

### Step b: Synthesis of tert-butyl [2-(1H-imidazol-1-yl)ethyl]carbamate

Sodium hydride (2.23 g, 56 mmol) was added slowly to the precooled dimethylformamide (40 ml) followed by the addition of imidazole (4.55 g, 66.2 mmol) at 0-5° C. The resulting reaction mixture was stirred for 10-15 minutes. The reaction mixture was brought to room temperature and stirred for 30 minutes followed by cooling to 0° C. To the reaction mixture was added the compound obtained from step a above (5 g, 22.3 mmol) and the mixture was stirred at room temperature overnight. The reaction mixture was diluted with water and stirred for 10-15 minutes followed by the addition of dichloromethane. The reaction mixture was stirred for 30 minutes. The organic layer was washed with water and brine, dried over anhydrous sodium sulphate and concentrated under reduced pressure to furnish the title compound. Yield: 2 g.

The following illustrative compound(s) were prepared analogously,

### Tert-butyl [2(2-isopropyl-1H-imidazol-1-yl)ethyl]carbamate

### Tert-butyl [2-(2-methyl-1H-imidazol-1-yl)ethyl]carbamate

### Synthesis of hydrochloride salt of 2-(1H-imidazol-1-yl)ethanamine (Formula III)

To the compound tent-butyl [2-(1H-imidazol-1-yl)ethyl]carbamate (2 g, 9.4 mmol) was added ethereal hydrochloric acid (15 ml) and stirred at room temperature for 2-3 hours. The solvent was evaporated under reduced pressure to furnish the title compound. Yield: 1.8 g.

The following illustrative compound(s) were prepared analogously,

### Hydrochloride salt of 2-(2-isopropyl-1H-imidazol-1-yl)ethanamine

### Hydrochloride salt of 2-(2-methyl-1H-imidazol-1-yl)ethanamine

### Synthesis of 3(2-methyl-1H-imidazol-1-yl)propan-1-amine (Formula III)

### Step a: Synthesis of 2-[3-(2-methyl-1H-imidazol-1-yl)propyl]-1H-isoindole-1,3(2H)-dione

To a solution of 2-methyl imidazole (306 mg, 3.7 mmol) and N-bromopropylpthalamide (1 g, 3.7 mmol) in dimethylformamide (50 ml) was added potassium carbonate (1.6 g, 11.2 mmol) and the reaction mixture was heated at 80° C. for 4 hours. The mixture was poured into water and extracted with ethyl acetate. The organic layer was washed with water and brine, dried over anhydrous sodium sulphate and concentrated under reduced pressure. The residue thus obtained was purified by column chromatography using 5% methanol in dichloromethane to furnish the title compound. Yield: 67 mg.

### Step b: Synthesis of 3-(2-methyl-1H-imidazol-1-yl)propan-1-amine

To a solution of the compound obtained from step a above (1 g, 3.7 mmol) in ethanol (20 ml) was added hydrazine hydrate (1 ml, 20 mmol) and heated the reaction mixture at 65-70° C. for 3 hours. The reaction mixture was cooled and filtered through celite pad and washed with ethanol. The filtrate was concentrated under reduced pressure. The residue thus obtained was diluted with dichloromethane and filtered through celite pad. The filtrate was concentrated under reduced pressure to furnish the title compound. Yield: 340 mg.

### Synthesis of hydrochloride salt of N-methyl-3-(2-methyl-1H-imidazol-1-yl)propan-1-amine (Formula III)

### Step a: Synthesis of tert-butyl [3-(2-methyl-1H-imidazol-1-yl)propyl]carbamate

A solution of the compound 3-(2-methyl-1H-imidazol-1-yl)propan-1-amine (2.7 g, 19.4 mmol) in dichloromethane (100 ml) was cooled at 0° C. followed by the dropwise addition of triethylamine (5.42 ml, 38.8 mmol). The reaction mixture was stirred at the same temperature for 30 minutes followed by the addition of tert-butoxycarbonyl anhydride (4.9 ml, 21.3 mmol). The reaction mixture was stirred at 0° C. for 1 hour and then at room temperature for 1 hour and 30 minutes. The aqueous solution of sodium bicarbonate was added to the reaction mixture and was extracted with dichloromethane. The organic layer was separated and washed with water and brine, dried over anhydrous sodium sulphate and concentrated under reduced pressure. The residue thus obtained was purified by column chromatography using 5% methanol in dichloromethane to furnish the title compound. Yield: 2.2 g.

### Step b: Synthesis of tent-butyl methyl[3-(2-methyl-1H-imidazol-1-yl)propyl]carbamate

To a solution of the compound obtained from step a above (500 mg, 2.1 mmol) in dry dimethylformamide (6 ml) was added sodium hydride (166 mg, 4.2 mmol) at 0° C. and stirred the reaction mixture for 30 minutes at the same temperature and then at room temperature for 30 minutes. The mixture was again cooled to 0° C. followed by the addition of iodomethane (0.2 ml, 2.5 mmol) in dimethylformamide (3 ml) and stirred at room temperature for 3 hours. Reaction mixture was quenched with water and extracted with ethyl acetate. The combined organic layer was washed with water and brine, dried over anhydrous sodium sulphate and concentrated under reduced pressure. The residue thus obtained was purified by column chromatography using 5% methanol in dichloromethane solvent mixture as eluent to furnish the title compound. Yield: 174 mg.

### Step c: Synthesis of hydrochloride salt of N-methyl-3-(2-methyl-1H-imidazol-1-yl)propan-1-amine

To a solution of the compound obtained from step b above (174 mg, 0.75 mmol) in dry diethyl ether (15 ml) was added slowly ethereal solution of hydrochloric acid (5 ml) and was stirred the mixture at room temperature overnight. The mixture was concentrated under reduce pressure to furnish the title compound. Yield: 147 mg.

### Synthesis of imidazol-1-yl-methanol

To a solution of the imidazole (1 g, 14.6 mmol) in dry tetrahydrofuran (30 ml) cooled to −10° C. was added butyl lithium (0.94 g, 14.6 mmol) dropwise at −30 to −20° C. The mixture was stirred for 30 minutes at −20° C. and subsequently cooled at −30° C. followed by the addition of paraformaldehyde (441 mg, 14.6 mmol). The reaction mixture was stirred for 30 minutes at −20° C. and then allowed to warm to room temperature. The resulting mixture was stirred overnight followed by the addition of water. The reaction mixture was extracted with ethyl acetate. The organic layer was dried over anhydrous sodium sulphate and concentrated under reduced pressure to furnish the title compound. Yield: 450 mg.

### Synthesis of methanesulphonic acid 2-(2-methyl-imidazol-1-yl)-ethyl ester

### Step a: Synthesis of (2-bromo-ethoxy)-tert-butyl-dimethyl-silane

To a solution of the 2-bromoethanol (5 g, 40 mmol) in dimethylformamide (25 ml) was added tert-butydimethylsilyl chloride (7.29 g, 48 mmol) and imidazole (6.86 g, 100 mmol). The reaction mixture was stirred overnight followed by quenching with water and extraction with ethyl acetate. The organic layer was dried over anhydrous sodium sulphate and concentrated under reduced pressure to furnish the title compound. Yield: 8 g.

### Step b: Synthesis of 1-[2-(tert-butyl-dimethyl-silanyloxy)-ethyl]-2-methyl-1H-imidazole

Sodium hydride (8.4 g, 210 mmol) was added slowly to dry dimethylformamide (40 ml) precooled at −10° C. under nitrogen atmosphere. To the resulting suspension was added 2-methyl imidazole (20.67 g, 252 mmol) at −10° C. and the reaction mixture was allowed to warm to room temperature. The reaction mixture was stirred for 1 hour at room temperature followed by cooling to 0° C. To the mixture was added solution of the compound obtained from step a above (20 g, 84 mmol) in dimethylformamide (10 ml) and stirred overnight at room temperature. The mixture was quenched with aqueous ammonium chloride solution and extracted with dichloromethane. The dichloromethane layer was dried over anhydrous sodium sulphate and concentrated under reduced pressure to furnish the title compound.

Yield: 12.5 g.

### Step c: Synthesis of 2-(2-methyl-imidazol-1-yl)-ethanol

To a compound obtained from step b above (12.5 g, 55.6 mmol) was added a solution of ethanolic hydrochloric acid solution (2%, 90 ml) at room temperature and the mixture was stirred overnight. The reaction mixture was concentrated under reduced pressure. The residue thus obtained washed with diethyl ether to furnish the title compound. Yield: 3.9 g.

### Step d: Synthesis of methanesulphonic acid 2-(2-methyl-imidazol-1-yl)-ethyl ester

To a solution of the compound obtained from step c above (4 g, 24.6 mmol) in dichloromethane (60 ml) was added triethylamine (10.27 ml, 73.8 mmol) and dimethylaminopyridine (150 mg) at 0° C. The reaction mixture was stirred until the compound obtained from step c above becomes completely soluble. The mixture was cooled down to −5° C. followed by the addition methane sulphonyl chloride (2.86 ml, 36.9 mmol) dropwise with stirring. The mixture was stirred for 3 hours at −5° C. and then overnight at room temperature. The mixture was diluted with sodium bicarbonate solution and extracted with dichloromethane. The dichloromethane layer was dried over anhydrous sodium sulphate and concentrated under reduced pressure to furnish the title compound. Yield: 3.8 g.

### Example 1

### Synthesis of 2-Cyclopentyl-2-hydroxy-N-[2-(1H-imidazol-1-yl)ethyl]-2-phenylacetamide (Compound No. 1)

To a solution of the hydrochloride salt of 2-(1H-imidazol-1-yl)ethanamine (0.5 g, 4.50 mmol) in chloroform (10 ml) was added N-methyl morpholine (2.96 ml, 27.02 mmol) and stirred the mixture for 5 to 10 minutes at the room temperature followed by the addition of 2-cyclopentyl-2-hydroxy-2-phenyl acetic acid (0.99 g, 4.5 mmol) and hydroxy benzotriazole (0.60 g, 4.5 mmol) at room temperature. The resulting reaction mixture was stirred for 30-45 minutes followed by the addition of 1-(3-dimethylaminopropyl)-3-ethylcarbodiimide hydrochloride and again stirred for over night. The mixture was diluted with water and stirred for 10-15 minutes followed by the addition of dichloromethane. The mixture was stirred for 15-20 minutes. The organic layer was separated, washed with water and brine, dried over anhydrous sodium sulphate and concentrated under reduced pressure. The residue thus obtained was purified by preparative column chromatography using 10% methanol in dichloromethane as eluent to furnish the title compound. Yield: 70 mg.

¹H NMR (CDCl₃) δ: 7.60-7.31 (5H, m), 6.99 (1H, s), 6.99 (1H, s), 6.65 (1H, s), 4.01-4.00 (2H, m), 3.99-3.49 (2H, m), 3.11-3.07 (1H, m), 1.64-1.42 (8H, m).

The following illustrative compounds were prepared analogously,

### 2-(4-Fluorophenyl)-2-hydroxy-N-[2-(1H-imidazol-1-yl)ethyl]-2-phenylacetamide (Compound No. 3)

¹H NMR (CD₃OD) δ: 7.45-7.29 (8H, m), 7.04-6.97 (4H, m), 4.18-4.15 (2H, m), 3.64-3.61 (2H, m).

### 2-Cyclobutyl-2-hydroxy-N-[2-(1H-imidazol-1-yl)ethyl]-2-phenylacetamide (Compound No. 4)

¹H NMR (CDCl₃) δ: 7.50-7.24 (6H, m), 6.94 (1H, s), 6.81 (1H, s), 4.01-3.98 (2H, m), 3.54-3.42 (3H, m), 2.04-1.72 (6H, m).

### 2-Cyclopentyl-2-(4-fluorophenyl)-2-hydroxy-N-[2-(1H-imidazol-1-yl)ethyl]acetamide (Compound No. 5)

¹H NMR (CD₃OD) δ: 7.59-7.44 (3H, m), 7.04-7.02 (2H, m), 6.94 (1H, s), 6.88 (1H, s), 4.08-4.05 (2H, m), 3.50-3.47 (2H, m), 3.05-3.01 (1H, m), 1.68-1.21 (8H, m).

### 2-Hydroxy-N-[2-(1H-imidazol-1-yl)ethyl]-2,2-diphenylacetamide (Compound No. 6)

¹H NMR (CD₃OD) δ: 7.58-7.27 (11H, m), 7.01 (s, 1H), 6.94 (s, 1H), 4.17-4.14 (2H, m), 3.63-3.60 (2H, m).

### 2-Cyclohexyl-2-hydroxy-N-[2-(1H-imidazol-1-yl)ethyl]-2-phenylacetamide (Compound No. 7)

¹H NMR (MeOD) δ: 7.66-7.53 (3H, m), 7.32-7.21 (3H, m), 6.91-6.89 (2H, m), 4.08-4.06 (2H, m), 3.50-3.30 (2H, m), 2.36 (1H, q), 1.60-1.10 (10H, m).

### 2-Cyclopentyl-2-hydroxy-N-[2-(2-isopropyl-1H-imidazol-1-yl)ethyl]-2-phenylacetamide (Compound No. 8)

¹H NMR (MeOD) δ: 7.59-7.57 (2H, m), 7.31-7.20 (3H, m), 6.74-6.73 (2H, m), 4.01-3.96 (2H, m), 3.47-3.30 (2H, m), 3.06-3.02 (2H, m), 1.57-1.51 (6H, m), 1.24-1.17 (8H, m).

### 2-Cyclohexyl-2-hydroxy-N-[2-(2-isopropyl-1H-imidazol-1-yl)ethyl]-2-phenylacetamide (Compound No. 9)

¹H NMR (MeOD) δ: 7.58-7.56 (2H, m), 7.36-7.20 (3H, m), 6.73-6.71 (2H, m), 3.99-3.96 (2H, m), 3.47-3.43 (2H, m), 3.29-3.01 (1H, m), 1.66-1.64 (1H, q), 1.40-1.17 (16H, m).

### 2-Hydroxy-N-[2-(2-isopropyl-1H-imidazol-1-yl)ethyl]-2-phenyl-2-pyridin-3-ylacetamide (Compound No. 10)

¹H NMR (CD₃OD) δ: 8.58-8.57 (1H, m), 8.46-8.44 (1H, m), 7.80-7.33 (1H, m), 7.33-7.26 (6H, m), 6.85-6.79 (2H, m), 4.11-4.07 (2H, m), 3.63-3.60 (2H, m), 3.29-3.07 (1H, m), 1.24-1.21 (6H, m).

### 2-(4-Fluorophenyl)-2-hydroxy-N-[2-(2-isopropyl-1H-imidazol-1-yl)ethyl]-2-phenylacetamide (Compound No. 11)

¹H NMR (CD₃OD) δ: 7.36-7.28 (7H, m), 7.03-6.98 (2H, m), 6.85-6.80 (2H, m), 4.10-4.06 (2H, m), 3.61-3.58 (2H, m), 3.12-3.07 (1H, m), 1.28-1.21 (6H, m).

### 2-Hydroxy-N-[2-(2-isopropyl-1H-imidazol-1-yl)ethyl]-2,2-diphenylacetamide (Compound No. 12)

¹H NMR (CD₃OD) δ: 7.35-7.27 (10H, m), 6.83-6.79 (2H, m), 4.09-4.06 (2H, m), 3.60-3.57 (2H, m), 3.29-3.06 (1H, m), 1.29-1.21 (6H, m).

### (2R)-2-cyclopentyl-2-hydroxy-N-[2-(2-methyl-1H-imidazol-1-yl)ethyl]-2-phenylacetamide (Compound No. 16)

¹H NMR (MeOD) δ: 7.54-7.52 (2H, m), 7.33-7.26 (5H, m), 4.19-4.08 (2H, m), 3.42-3.30 (2H, m), 3.04-2.90 (1H, m), 1.56-1.47 (8H, m).

### 2-Hydroxy-N-[2-(1H-imidazol-1-yl)ethyl]-2-phenyl-2-pyridin-3-ylacetamide (Compound no. 17)

¹H NMR (CDCl₃) δ: 8.49 (s, 1H), 8.03-8.08 (m, 3H), 7.70-7.72 (m, 1H), 7.44-7.45 (m, 2H), 7.14-7.33 (m, 3H), 6.89-6.92 (m, 2H), 6.82 (s, 1H), 4.02-4.11 (m, 2H), 3.47-3.67 (m, 2H).

### 2-Cyclopentyl-2-hydroxy-N-[2-(2-methyl-1H-imidazol-1-yl)ethyl]-2-phenylacetamide (Compound No. 18)

¹H NMR (CDCl₃) δ: 7.58-7.60 (m, 2H), 7.26-7.37 (m, 3H), 6.88 (brs, 1H), 6.79 (s, 1H), 6.48 (s, 1H), 3.86-3.91 (s, 2H), 3.46-3.51 (s, 2H), 3.07-3.11 (q, 1H), 2.20 (s, 3H), 1.14-1.68 (m, 8H).

### 2-Hydroxy-N-[2-(2-methyl-1H-imidazol-1-yl)ethyl]-2-phenyl-2-pyridin-3-ylacetamide (Compound No. 19)

¹H NMR (CDCl₃) δ: 8.48 (s, 1H), 8.13-8.15 (m, 1H), 7.71-7.75 (m, 2H), 7.16-7.44 (m, 5H), 6.62 (s, 1H), 6.55 (s, 1H), 3.68-3.99 (m, 2H), 3.54-3.66 (m, 2H), 2.06 (s, 3H).

### 2-Cyclohexyl-2-hydroxy-N-[2-(2-methyl-1H-imidazol-1-yl)ethyl]-2-phenylacetamide (Compound No. 20)

¹H NMR (CDCl₃) δ: 7.57-7.59 (m, 2H), 7.29-7.38 (m, 3H), 6.94 (brs, 1H), 6.82 (s, 1H), 6.49 (s, 1H), 3.86-3.92 (m, 2H), 3.47-3.51 (m, 2H), 2.42-2.45 (m, 1H), 2.21 (s, 3H), 0.83-1.82 (m, 10H).

### 2-(4-Fluorophenyl)-2-hydroxy-N-[2-(2-methyl-1H-imidazol-1-yl)ethyl]-2-phenylacetamide (Compound No. 21)

¹H NMR (CDCl₃) δ: 7.32-7.43 (m, 7H), 7.00-7.03 (m, 2H), 6.57 (s, 1H), 6.51 (s, 1H), 3.95-3.98 (m, 2H), 3.59-3.63 (m, 2H), 2.15 (s, 3H).

### 2-Cyclopentyl-2-(4-fluorophenyl)-2-hydroxy-N-[2-(2-methyl-1H-imidazol-1-yl)ethyl]acetamide (Compound No. 22)

¹H NMR (CDCl₃) δ: 7.57-7.60 (m, 2H), 7.00-7.04 (m, 2H), 6.82 (s, 1H), 6.57 (s, 1H), 3.91-3.94 (m, 2H), 3.48-3.53 (m, 2H), 3.04-3.06 (q, 1H), 2.25 (s, 3H), 1.42-1.64 (m, 8H).

### 2-Cyclobutyl-2-hydroxy-N-[2-(2-methyl-1H-imidazol-1-yl)ethyl]-2-phenylacetamide (Compound No. 23)

¹H NMR (CDCl₃) δ: 7.50-7.52 (m, 2H), 7.27-7.36 (m, 3H), 6.88 (brs, 1H), 6.78 (s, 1H), 6.54 (s, 1H), 3.89-3.93 (m, 2H), 3.48-3.50 (m, 2H), 3.43-3.47 (m, 1H), 2.23 (s, 3H), 1.73-2.06 (m, 6H).

### 2-Hydroxy-N-[2-(2-methyl-1H-imidazol-1-yl)ethyl]-2,2-diphenylacetamide (Compound No. 24)

¹H NMR (CDCl₃) δ: 7.09-7.42 (m, 10H), 6.59 (s, 1H), 6.55 (s, 1H), 3.95-3.97 (m, 2H), 3.58-3.64 (m, 2H), 2.17 (s, 3H).

### 3,3,3-Trifluoro-2-hydroxy-N-[2-(2-methyl-1H-imidazol-1-yl)ethyl]-2-(4-methylphenyl)propanamide (Compound No. 25)

¹H NMR (CD₃OD) δ: 7.47-7.49 (m, 2H), 7.17-7.20 (m, 2H), 6.81 (s, 1H), 6.73 (s, 1H), 3.99-4.02 (m, 2H), 3.41-3.65 (m, 2H), 2.34 (s, 3H), 2.21 (s, 3H).

### N-[2-(2-methyl-1H-imidazol-1-yl)ethyl]-2,2-diphenylacetamide (Compound No. 26)

¹H NMR (CD₃OD) δ: 7.21-7.31 (m, 10H), 6.83 (s, 1H), 6.76 (s, 1H), 4.00-4.03 (m, 2H), 3.52-3.55 (m, 2H), 2.17 (s, 3H).

### 2-Cyclopentyl-N-[2-(2-methyl-1H-imidazol-1-yl)ethyl]-2-phenylacetamide (Compound No. 27)

¹H NMR (CD₃OD) δ: 7.22-7.31 (5H, m), 6.71 (s, 1H), 6.63 (s, 1H), 3.91-3.94 (m, 2H), 3.31-3.43 (m, 2H), 2.42-2.45 (m, 1H), 2.18 (s, 3H), 1.65-1.95 (m, 8H).

### 2-Cyclopentyl-2-N-[2-(2-methyl-1H-imidazol-1-yl)ethyl]-2-(4-methylphenyl)acetamide (Compound No. 28)

¹H NMR (MeOD) δ: 7.43-7.41 (2H, m), 7.12-7.10 (2H, m), 6.76 (1H, s), 6.71 (1H, s), 3.98-3.92 (2H, m), 3.46-3.44 (2H, m), 3.29 (1H, m), 2.31-2.30 (3H, m), 2.28-2.16 (3H, m), 1.53-1.24 (8H, m).

### 2-Cyclohexyl-2-hydroxy-N-[2-(2-methyl-1H-imidazol-1-yl)ethyl]-2-(4-methylphenyl)acetamide (Compound No. 29)

¹H NMR (MeOD) δ: 7.43-7.41 (2H, m), 7.12-7.10 (2H, m), 6.75 (1H, s), 6.70 (1H, s), 3.98-3.91 (2H, m), 3.47-3.44 (2H, m), 2.30-2.29 (4H, m), 2.16-2.15 (3H, s), 1.30-1.19 (10, m).

### 2-Hydroxy-N-[2-(2-methyl-1H-imidazol-1-yl)ethyl]-2-(4-methylphenyl)-2-phenylacetamide (Compound No. 30)

¹H NMR MeOD) δ: 7.35-7.27 (5H, m), 7.26-7.20 (2H, m), 7.18-7.09 (2H, m), 6.84 (1H, s), 6.75 (1H, s), 4.05-4.04 (2H, m), 3.60-3.57 (2H, m), 2.31-2.28 (3H, m), 2.22-2.15 (3H, m).

### 2-Cyclopentyl-2-hydroxy-N-[3-(1H-imidazol-1-yl)propyl]-2-phenylacetamide (Compound No. 31)

¹H NMR (CDCl₃) δ: 7.28-7.63 (m, 5H), 7.03 (s, 1H), 6.84 (s, 1H), 6.62 (s, 1H), 3.78-3.82 (t, 2H), 3.19-3.24 (t, 2H), 3.09 (m, 1H), 0.88-1.94 (m, 10H).

### 2-Cyclohexyl-2-hydroxy-N-[3-(1H-imidazol-1-yl)propyl]-2-phenylacetamide (Compound No. 32)

¹H NMR (CDCl₃) δ: 7.28-7.62 (m, 5H), 7.04 (s, 1H), 6.83 (s, 1H), 6.75 (s, 1H), 3.77-3.80 (t, 2H), 3.20-3.23 (t, 2H), 2.42-2.45 (m, 1H), 0.88-1.94 (m, 12H).

### 2-Cyclopentyl-2-hydroxy-N-[3-(2-methyl-1H-imidazol-1-yl)propyl]-2-phenylacetamide (Compound No. 33)

¹H NMR (CDCl₃) δ: 7.27-7.63 (m, 5H), 6.74 (m, 1H), 6.64 (s, 1H), 6.22 (s, 1H), 3.71-3.74 (t, 2H), 3.23-3.26 (t, 2H), 3.09 (m, 1H), 2.24 (s, 3H), 1.48-1.88 (m, 10H).

### 2-Cyclohexyl-2-hydroxy-N-[3-(2-methyl-1H-imidazol-1-yl)propyl]-2-phenylacetamide (Compound No. 34)

¹H NMR (CDCl₃) δ: 7.28-7.62 (m, 5H), 6.88 (s, 1H), 6.76 (s, 1H), 6.73 (s, 1H), 3.68-3.73 (t, 2H), 3.22-3.25 (t, 2H), 2.24 (m, 1H), 2.23 (s, 3H), 1.20-1.88 (m, 12H).

### (2R)-2-(3,3-Difluorocyclopentyl)-2-hydroxy-N-[3-(2-methyl-1H-imidazol-1-yl)propyl]-2-phenylacetamide (Compound No. 35)

¹H NMR (CDCl₃) δ: 7.30-7.61 (m, 5H), 6.84 (s, 1H), 6.72 (s, 1H), 6.71 (m, 1H), 3.69-3.73 (t, 2H), 3.21-3.35 (m, 3H), 2.23 (s, 3H), 1.52-2.18 (m, 8H).

### 2-Cyclopentyl-2-hydroxy-N-methyl-N-[3-(2-methyl-1H-imidazol-1-yl)propyl]-2-phenylacetamide (Compound No. 36)

¹H NMR (CDCl₃) δ: 7.31-7.36 (m, 5H), 6.9 (s, 1H), 6.7 (s, 1H), 3.78 (bs, 2H), 3.35 (bs, 2H), 2.73 (bs, 3H), 2.29 (s, 4H), 1.25-1.17 (m, 10H).

### 2-Cyclohexyl-2-hydroxy-N-methyl-N-[3-(2-methyl-1H-imidazol-1-yl)propyl]-2-phenylacetamide (Compound No. 37)

¹H NMR (CDCl₃) δ: 7.26-7.36 (m, 5H), 6.91 (s, 1H), 6.73 (s, 1H), 3.7-3.8 (bs, 2H), 3.33 (bs, 2H), 2.8 (s, 3H), 2.27 (s, 3H), 1.25-1.82 (m, 13H).

### 2-Cyclopentyl-2-hydroxy-N-[2-(1H-imidazol-1-yl)ethyl]-N-methyl-2-phenylacetamide (Compound No. 38)

¹H NMR (CDCl₃) δ: 7.29-7.77 (m, 6H), 7.00 (s, 1H), 6.77 (s, 1H), 4.11 (bs, 1H), 3.73 (bs, 1H), 3.49 (bs, 1H), 2.94-2.98 (m, 1H), 2.38-2.50 (m, 4H), 1.14-1.66 (m, 8H).

### 2-Cyclohexyl-2-hydroxy-N-[2-(1H-imidazol-1-yl)ethyl]-N-methyl-2-phenylacetamide (Compound No. 57)

¹H NMR (CDCl₃) δ: 7.19-7.43 (m, 5H), 6.96 (s, 1H), 4.04 (bs, 2H), 3.49-3.68 (bd, 2H), 2.60 (s, 3H), 2.38-2.45 (m, 1H, 1.17-1.63 (m, 10H).

### 2-Cyclopentyl-2-hydroxy-N-[2-(1H-imidazol-4-yl)ethyl]-2-phenylacetamide (Compound No. 39)

¹H NMR (MeOD) δ: 7.59-7.57 (3H, m), 7.31-7.20 (3H, m), 6.69 (1H, s), 3.43-3.34 (2H, m), 3.08 (1H, q), 2.73-2.70 (2H, m), 1.62-1.25 (8H, m).

### 2-Cyclopentyl-2-hydroxy-N-[2-(1H-imidazol-4-yl)ethyl]-2-(4-methylphenyl)acetamide (Compound No. 41)

¹H NMR (CD₃OD) δ: 7.60 (s, 1H), 7.43-7.45 (dd, 2H, J=8 Hz), 7.09-7.11 (dd, 2H, J=8 Hz), 6.71 (s, 1H), 3.34-3.44 (m, 2H), 3.30 (q, 1H), 2.70-2.73 (m, 2H), 1.49-1.62 (m, 8H).

### 2-Cyclohexyl-2-hydroxy-N-[2-(1H-imidazol-4-yl)ethyl]-2-(4-methylphenyl)acetamide (Compound No. 42)

¹H NMR (CD₃OD) δ: 7.59 (s, 1H), 7.42-7.44 (dd, 2H, 8 Hz), 7.09-7.11 (dd, 2H, 8 Hz), 6.69 (s, 1H), 3.30-3.41 (m, 2H), 2.68-2.72 (m, 2H), 2.31 (m, 1H), 2.29 (s, 1H), 1.29-1.74 (m, 10H).

### 2-Hydroxy-N-[2-(1H-imidazol-4-yl)ethyl]-2,2-diphenylacetamide (Compound No. 43)

¹H NMR (CD₃OD) δ: 7.59 (s, 1H), 7.27-7.38 (m, 10H), 6.75 (s, 1H), 3.49-3.53 (m, 2H), 2.78-2.81 (m, 2H).

### 2-Cyclopentyl-2-hydroxy-N-[2-(1H-imidazol-1-yl)ethyl]-N-methyl-2-phenylacetamide (Compound No. 58)

¹H NMR (CD₃OD) δ: 7.27-7.42 (m, 5H), 6.83 (s, 1H), 6.60 (s, 1H), 3.96 (bs, 2H), 3.48-3.65 (bd, 2H), 2.97 (m, 1H), 2.62 (s, 3H), 2.30 (s, 3H), 1.5-1.69 (m, 8H).

### Example 2

### Synthesis of 1H-imidazol-1-ylmethyl cyclopentyl(hydroxy)phenylacetate (Compound No. 46)

To a solution of 2-cyclopentyl-2-hydroxy-2-phenyl acetic acid (400 mg, 1.8 mmol) in dry tetrahydrofuran (20 ml) under argon atmosphere was added carbonyldiimidazole (294 mg, 1.8 mmol) and stirred the mixture for 5 min. To the resulting reaction mixture was added imidazol-1yl-methanol (178 mg, 1.8 mmol) and stirred for 24 hours. The mixture was concentrated under reduced pressure and the residue thus obtained was washed with water and extracted with dichloromethane. The organic layer was washed with water and brine, dried over anhydrous sodium sulphate and concentrated under reduced pressure. The residue thus obtained was purified by column chromatography using 5% methanol in dichloromethane to furnish the title compound. Yield: 140 mg.

¹H NMR (CD₃OD) δ: 7.68 (s, 1H), 7.55-7.57 (m, 2H), 7.28-7.34 (m, 3H), 7.04-7.11 (m, 2H), 5.76-6.06 (m, 2H), 2.77-2.86 (m, 1H), 1.27-1.62 (m, 8H).

The following illustrative compounds were prepared analogously by coupling the appropriate acid (racemic or pure isomers, as applicable in each case) with an appropriate alcohol.

### 1H-Imidazol-1-ylmethyl cyclohexyl(hydroxy)(4-methylphenyl)acetate (Compound No. 2)

¹H NMR (CDCl₃) δ: 7.72 (1H, s), 7.42-7.40 (2H, m), 7.16-7.03 (4H, m), 6.01-5.98 (1H, m), 5.81-5.78 (1H, m), 2.40-2.32 (3H, m), 2.04-2.02 (1H, m), 1.67-1.25 (10H, m).

### 1H-Imidazol-1-ylmethyl cyclohexyl(hydroxy)phenylacetate (Compound No. 47)

¹H NMR (CDCl₃) δ: 7.68 (s, 1H), 7.54-7.56 (m, 2H), 7.27-7.34 (m, 3H), 6.97-7.05 (m, 2H), 5.80-6.97 (m, 2H), 2.16-2.18 (m, 1H), 0.88-1.62 (m, 10H).

### 1H-Imidazol-1-ylmethyl (2R)-cyclopentyl(hydroxy)phenylacetate (Compound No. 48)

¹H NMR (CDCl₃) δ: 7.82 (s, 1H), 7.53-7.55 (m, 2H), 7.21-7.30 (m, 4H), 6.94 (s, 1H), 5.95-6.09 (m, 2H), 2.87-2.89 (m, 1H), 1.28-1.53 (m, 8H).

### 1H-Imidazol-1-ylmethyl cyclopentyl(hydroxy)(4-methoxyphenyl)acetate (Compound No. 49)

¹H NMR (CDCl₃) δ: 7.68 (1H, s), 7.48-7.45 (2H, m), 7.07-7.04 (2H, m), 6.86-6.83 (2H, m), 6.05-6.03 (1H, m), 5.78-5.75 (1H, m), 3.82 (3H, s), 2.85-2.76 (1H, q), 1.62-1.25 (8H, m).

### Example 3

### Synthesis of 2-(2-methyl-1H-imidazol-1-yl)ethyl cyclopentyl(hydroxy)phenylacetate (Compound No. 13)

To a solution of the compound 2-cyclopentyl-2-hydroxy-2-phenyl acetic acid (388 mg, 1.7 mmol), methanesulphonic acid 2-(2-methyl-imidazol-1-yl)-ethyl ester (300 mg, 1.4 mmol) and toluene (15 ml) was added 1,8-diazabicyclo[5.4.0]undecen-7-ene (447 mg, 2.9 mmol) and stirred the mixture overnight under reflux. The organic solvent was evaporated under reduced pressure and the residue thus obtained was purified by column chromatography using 5% methanol in dichloromethane. Yield: 60 mg.

¹H NMR (CDCl₃) δ: 7.52-7.54 (m, 2H), 7.28-7.35 (m, 3H), 6.90 (s, 1H), 6.71 (s, 1H), 4.36-4.39 (m, 2H), 4.06-4.09 (m, 2H), 2.81-2.85 (q, 1H), 2.31 (s, 3H), 1.33-1.57 (m, 8H).

The following illustrative compound(s) were prepared analogously by coupling the appropriate acid (racemic or pure isomers, as applicable in each case) with an appropriate ester.

### 2(2-Methyl-1H-imidazol-1-yl)ethyl (2R)-cyclopentyl(hydroxy)phenylacetate (Compound No. 14)

¹H NMR (CDCl₃) δ: 7.51-7.53 (m, 2H), 7.30-7.44 (m, 3H), 6.92 (s, 1H), 6.56 (s, 1H), 4.31-4.41 (9m, 2H), 4.06-4.09 (m, 2H), 2.81-2.86 (m, 1H), 2.36 (s, 3H), 1.32-1.66 (m, 8H).

### 2-(2-Methyl-1H-imidazol-1-yl)ethyl cyclopentyl(phenyl)acetate (Compound No. 59)

¹H NMR (CDCl₃) δ: 7.31-7.22 (5H, m), 6.82 (1H, s), 6.50 (1H, s), 4.25-4.21 (2H, m), 3.97-3.93 (2H, m), 2.25-2.51 (2H, m), 2.27 (3H, s), 1.94-1-90 (4H, m), 1.74-1.56 (4H, m).

### 2(2-Methyl-1H-imidazol-1-yl)ethyl cyclohexyl(hydroxy)phenylacetate (Compound No. 60)

¹H NMR (CDCl₃) δ: 7.51-7.49 (2H, m), 7.34-7.22 (3H, m), 6.91 (1H, s), 6.59 (1H, s), 4.40-4.36 (2H, m), 4.08-4.05 (2H, m), 2.32 (3H, s), 2.17-2.12 (1H, m), 1.78-1.65 (5H, m), 1.42-1.11 (5H, m).

### 3-(2-Methyl-1H-imidazol-1-yl)propyl cyclopentyl(hydroxy)phenylacetate (Compound No. 61)

¹H NMR (CD₃OD) δ: 7.63-7.65 (m, 2H), 7.25-7.37 (m, 3H), 6.79-6.81 (m, 2H), 4.06-4.09 (t, 2H), 3.82-3.85 (t, 2H), 2.48 (m, 1H), 2.18 (s, 3H), 2.01 (q, 2H), 0.7-1.7 (m, 8H).

### 3-(2-Methyl-1H-imidazol-1-yl)propyl (2R)-[(1R)-3,3-difluorocyclopentyl](hydroxy)phenylacetate (Compound No. 62)

¹H NMR (CD₃OD) δ: 7.62-7.63 (m, 2H), 7.28-7.61 (m, 3H), 6.78-6.79 (m, 2H), 4.08-4.88 (t, 2H), 3.83-3.86 (t, 2H), 3.24-3.25 (m, 1H), 2.17 (s, 3H), 1.28-2.06 (m, 8H).

### 3-(2-Methyl-1H-imidazol-1-yl)propyl 2R-2-(1S or 1R) (3,3-difluorocyclohexyl)(hydroxy)phenylacetate (Compound No. 63)

¹H NMR (CD₃OD) δ: 7.63-7.64 (m, 2H), 7.30-7.61 (m, 3H), 6.76-6.77 (m, 2H), 4.09-4.13 (m, 2H), 3.81-3.86 (m, 2H), 2.6 (m, 1H), 2.16 (s, 3H), 1.2-2.04 (m, 10H).

### 2(2-Isopropyl-1H-imidazol-1-yl)ethyl cyclohexyl(hydroxy)phenylacetate (Compound No. 64)

¹H NMR (CDCl₃) δ: 7.50-7.52 (m, 2H), 7.26-7.35 (m, 3H), 6.94 (s, 1H), 6.63 (s, 1H), 4.36-4.41 (m, 2H), 4.11-4.14 (m, 2H), 2.94-2.98 (q, 1H), 2.15 (q, 1H), 1.09-1.65 (q, 16H).

### 2-(1H-Imidazol-1-yl)ethyl cyclopentyl(hydroxy)phenylacetate (Compound No. 65)

¹H NMR (CD₃OD) δ: 7.50-7.53 (m, 3H), 7.24-7.32 (m, 3H), 6.92-6.98 (m, 2H), 4.34-4.37 (m, 2H), 4.25-4.28 (m, 2H), 2.85-2.89 (q, 1H), 1.28-1.56 (m, 8H).

### 2(2-Methyl-1H-imidazol-1-yl)ethyl (2R)-[(1S)-3,3-difluorocyclopentyl](hydroxy)phenylacetate (Compound No. 66)

¹H NMR (CD₃OD) δ: 7.45-7.47 (m, 2H), 7.25-7.40 (m, 3H), 6.99 (s, 1H), 6.61 (s, 1H), 4.20-4.59 (m, 4H), 3.07 (q, 1H), 2.53 (s, 3H), 1.44-2.25 (m, 6H).

### 2-(1H-Imidazol-1-yl)ethyl cyclohexyl(hydroxy)phenylacetate (Compound No. 67)

¹H NMR (CDCl₃) δ: 7.28-7.75 (m, 6H), 7.05 (s, 1H), 6.75 (s, 1H), 4.40-4.43 (m, 2H), 4.15-4.19 (m, 2H), 1.08-2.20 (m, 11H).

### 2(2-Isopropyl-1H-imidazol-1-yl)ethyl cyclopentyl(hydroxy)phenylacetate (Compound No. 68)

¹H NMR (CDCl₃) δ: 7.53-7.69 (m, 2H), 7.27-7.35 (m, 3H), 6.93 (s, 1H), 6.60 (s, 1H), 4.36-4.40 (m, 2H), 4.10-4.12 (m, 2H), 2.93-2.96 (m, 1H), 2.81-2.85 (m, 1H), 1.30-1.56 (m, 16H).

### 2-(2-Methyl-1H-imidazol-1-yl)ethyl (2R)-[(1R)-3,3-difluorocyclopentyl](hydroxy)phenylacetate (Compound No. 69)

¹H NMR (CD₃OD) δ: 7.47-7.49 (m, 2H), 7.32-7.37 (m, 3H), 6.92 (s, 1H), 6.60 (s, 1H), 4.40-4.51 (m, 2H), 4.14-4.18 (m, 2H), 3.08-3.10 (q, 1H), 2.43 (s, 3H), 1.64-2.27 (m, 6H).

### 2-(2-Isopropyl-1H-imidazol-1-yl)ethyl cycloheptyl(hydroxy)phenylacetate (Compound No. 70)

¹H NMR (CD₃OD) δ: 7.45-7.47 (m, 2H), 7.23-7.30 (m, 3H), 6.82-6.85 (m, 2H), 4.22-4.37 (m, 4H), 3.11-3.13 (q, 1H), 2.3 (m, 1H), 1.22-1.55 (m, 18H).

### 2-(2-Methyl-1H-imidazol-1-yl)ethyl cycloheptyl(hydroxy)phenylacetate (Compound No. 71)

¹H NMR (CD₃OD) δ: 7.45-7.48 (m, 2H), 7.23-7.31 (m, 3H), 6.88 (s, 1H), 6.77 (s, 1H), 4.18-4.37 (m, 4H), 2.35 (m, 1H), 2.30 (s, 3H), 1.21-1.81 (m, 12H).

### 3-(2-Methyl-1H-imidazol-1-yl)propyl (2R)-[(1S)-3,3-difluorocyclopentyl](hydroxy)phenylacetate (Compound No. 110)

¹H NMR (CD₃OD) δ: 7.63-7.64 (m, 2H), 7.28-7.40 (m, 3H), 6.78-6.80 (m, 2H), 4.08-4.09 (t, 2H), 3.82-3.87 (t, 2H), 3.22-3.24 (m, 1H), 2.17 (s, 3H), 1.28-2.16 (m, 8H).

### 2-(2-Isopropyl-1H-imidazol-1-yl)ethyl (2R)-[(1S)-3,3-difluorocyclopentyl](hydroxy)phenylacetate (Compound No. 112)

¹H NMR (CD₃OD) δ: 7.32-7.47 (m, 2H), 7.26-7.32 (m, 3H), 6.80-6.81 (m, 2H), 4.36-4.40 (m, 2H), 4.23-4.26 (m, 2H), 3.06-3.11 (m, 2H), 1.23-2.06 (m, 12H).

### 2-(2-Isopropyl-1H-imidazol-1-yl)ethyl (2R)-[(1R)-3,3-difluorocyclopentyl](hydroxy)phenylacetate (Compound No. 113)

¹H NMR (CD₃OD) δ: 7.45-7.48 (m, 2H), 7.26-7.33 (m, 3H), 6.85 (s, 1H), 6.83 (s, 1H), 4.36-4.42 (m, 2H), 4.25-4.28 (m, 2H), 3.10-3.12 (m, 2H), 1.24-2.06 (m, 12H).

### Example 4

### Synthesis of N-[2-(1-benzyl-1H-imidazol-4-yl)ethyl]-2-cyclopentyl-2-hydroxy-2-(4-methylphenyl)acetamide (Compound No. 40)

To a solution of the Compound No. 41 (0.15 g, 0.45 mmol) in methanol (2 ml) and acetone (15-20 ml) was added potassium carbonate (0.189 g, 1.37 mmol) and tetra-butyl ammonium bromide (catalytic amount) and stirred the mixture at room temperature for 1 hour. To the resulting mixture was added benzyl bromide (0.054 ml, 0.00045 mol) and stirred at room temperature for overnight. The reaction mixture was filtered and the filtrate was concentrated under reduced pressure. The residue thus obtained was purified by column chromatography using 5% methanol in dichloromethane as an eluent to furnish the title compound. Yield: 80 mg.

¹H NMR (CD₃OD) δ: 7.48-7.46 (3H, m), 7.39-7.37 (3H, m), 7.15-7.09 (4H, m), 6.52 (1H, s), 5.01 (2H, s), 3.45-3.42 (2H, m), 2.95-2.93 (1H, q), 2.69-2.63 (2H, m), 2.30 (3H, m), 1.60-1.27 (8H, m).

The following illustrative compounds were prepared similarly,

### N-[2-(1-benzyl-1H-imidazol-4-yl)ethyl]-2-cyclohexyl-2-hydroxy-2-(4-methylphenyl)acetamide (Compound No. 44)

¹H NMR (CDCl₃) δ: 7.09-7.48 (m, 10H), 6.50 (s, 1H), 5.01 (s, 2H), 3.40-3.48 (m, 2H), 2.64-2.68 (m, 2H), 2.30 (s, 3H), 1.65-1.71 (m, 1H), 0.95-1.57 (m, 10H).

### N-[2-(1-benzyl-1H-imidazol-4-yl)ethyl]-2-hydroxy-2,2-diphenylacetamide (Compound No. 45)

¹H NMR (CDCl₃) δ: 7.08-7.41 (m, 16H), 6.49 (s, 1H), 4.96 (s, 2H), 3.55-3.61 (m, 2H), 2.69-2.73 (m, 2H).

### Example 5

### Synthesis of 1-cyclohexyl-1-hydroxy-3-(1H-imidazol-1-yl)-1-phenylacetone (Compound No. 50)

### Step a: Synthesis of 1-cyclohexyl-1-hydroxy-1-phenylacetone

The compound methyl lithium (1.879 g, 85.4 mmol) was added dropwise to dry tetrahydrofuran (75 ml) under argon atmosphere at room temperature under constant stirring. To the resulting mixture was added a solution of 2-cyclohexyl-2-hydroxy-2-phenyl acetic acid (5 g, 21.36 mmol) in dry tetrahydrofuran (55 ml) slowly. The mixture was stirred at room temperature for 2 hours and then refluxed for approx. 3-4 hours. The reaction mixture was cooled followed by the addition of hydrochloric acid (10%, 500 ml) under constant stirring. The reaction mixture was extracted with ethyl acetate. The organic layer was concentrated under reduced pressure. The residue thus obtained was purified by column chromatography using 3% ethyl acetate in hexane to furnish the title compound. Yield: 2.7 g.

### Step b: Synthesis of 3-bromo-1-cyclohexyl-1-hydroxy-1-phenylacetone

To a solution of the compound obtained from step a above (2.7 g, 11.63 mmol) in dry tetrahydrofuran (30 ml) under argon atmosphere was added a solution of pyridinium tribromide (5.68 g, 15.1 mmol) in tetrahydrofuran (50 ml) dropwise over 2 hours. The reaction mixture was stirred for 24 hours. The solid thus obtained was filtered. The filtrate was concentrated under reduced pressure and the residue thus obtained was washed with water and extracted with ethyl acetate. The ethyl acetate layer was dried over anhydrous sodium sulphate and concentrated under reduced pressure. The residue thus obtained was purified by column chromatography to furnish the title compound. Yield: 2.2 g.

### Step c: Synthesis of 1-cyclohexyl-1-hydroxy-3-(1H-imidazol-1-yl)-1-phenylacetone (Compound No. 50)

To a solution of the compound imidazole (71 mg, 1.05 mmol) in dichloromethane (5 ml) was added triethylamine (0.25 ml, 1.77 mmol) followed by the addition of a solution of the compound obtained from step b above (250 mg, 0.8 mmol) in dichloromethane (5 ml) dropwise under constant stirring. The reaction mixture was stirred for overnight. The organic solvent was evaporated under reduced pressure and the residue thus obtained was purified by column chromatography using 5% methanol in dichloromethane.

¹H NMR (CDCl₃) δ: 7.31-7.54 (m, 6H), 7.01 (s, 1H), 6.64 (s, 1H), 4.86-5.02 (m, 2H), 2.43-2.46 (m, 1H), 0.99-1.82 (m, 10H).

The following illustrative analogues were prepared similarly,

### 1-Cyclopentyl-1-hydroxy-1-(4-methoxyphenyl)-3-(2-methyl-1H-imidazol-1-yl)acetone (Compound No. 15)

¹H NMR (CDCl₃) δ: 7.47-7.45 (2H, dd, J=8 Hz), 6.93-6.91 (2H, dd, J=8 Hz), 6.85 (1H, s), 6.56 (1H, s), 4.94-4.78 (2H, m), 3.82 (3H, s), 3.07-3.01 (1H, q), 2.17 (3H, s), 1.67-1.25 (8H, m).

### 1-Cyclohexyl-1-hydroxy-3-(2-methyl-1H-imidazol-1-yl)-1-phenylacetone (Compound No. 51)

¹H NMR (CDCl₃) δ: 7.30-7.55 (m, 5H), 6.85 (s, 1H), 6.56 (s, 1H), 4.95-4.99 (dd, 1H, 16 Hz), 4.80-4.84 (dd, 1H, 16 Hz), 2.41-2.47 (m, 1H), 1.82 (s, 3H), 1.03-1.70 (m, 10H).

### 1-Cyclopentyl-1-hydroxy-3-(2-isopropyl-1H-imidazol-1-yl)-1-(4-methoxyphenyl)acetone (Compound No. 52)

¹H NMR (CDCl₃) δ: 7.46-7.48 (2H, dd, 8 Hz), 6.92-6.94 (2H, dd, 8 Hz), 6.90 (s, 1H), 6.51 (s, 1H), 4.81-4.99 (m, 2H), 3.83 (s, 3H), 3.05-3.09 (m, 1H), 2.25-2.27 (m, 1H), 1.58-1.66 (m, 8H), 1.07-1.12 (m, 6H).

### 1-Cyclohexyl-1-hydroxy-3-(2-isopropyl-1H-imidazol-1-yl)-1-phenylacetone (Compound No. 53)

¹H NMR (CDCl₃) δ: 7.32-7.55 (m, 5H), 6.93 (s, 1H), 6.50 (s, 1H), 4.93-4.98 (dd, 1H, 20 Hz), 4.80-4.85 (dd, 1H, 20 Hz), 2.50 (m, 1H), 2.17-2.20 (m, 1H), 1.34-1.72 (m, 10H), 0.99-1.09 (m, 6H).

### 1-Cyclohexyl-1-hydroxy-3-(2-methyl-4,5-dihydro-1H-imidazol-1-yl)-1-phenylacetone (Compound No. 54)

¹H NMR (CDCl₃) δ: 7.49-7.51 (m, 2H), 7.29-7.39 (m, 3H), 4.39-5.04 (m, 2H), 3.85-3.90 (m, 2H), 3.60-3.65 (m, 2H), 2.24 (m, 1H), 2.07 (s, 3H), 1.10-1.81 (m, 10H).

### 1-Cyclopentyl-1-hydroxy-1-(4-methoxyphenyl)-3-(2-methyl-4,5-dihydro-1H-imidazol-1-yl)acetone (Compound No. 55)

¹H NMR (CDCl₃) δ: 7.43-7.45 (dd, 2H, 8 Hz), 6.88-6.90 (dd, 2H, 8 Hz), 4.41-5.07 (m, 2H), 3.80-3.88 (m, 2H), 3.65 (s, 3H), 3.17-3.19 (m, 2H), 2.93 (m, 1H), 2.16 (s, 3H), 1.31-1.63 (m, 8H).

### 1-Cyclopentyl-1-hydroxy-3-(1H-imidazol-1-yl)-1-(4-methoxyphenyl)acetone (Compound No. 56)

¹H NMR (CDCl₃) δ: 7.46-7.44 (2H, m), 7.27-7.25 (1H, m), 7.02 (1H, s), 6.94-6.92 (2H, m), 6.64 (1H, s), 4.88 (2H, s), 3.83 (3H, s), 3.05 (1H, q), 1.68-1.25 (8H, m).

### Example 6

### 3-Benzyl-1-(2-{[cycloheptyl(hydroxy)phenylacetyl]oxy}ethyl)-2-isopropyl-1H-imidazol-3-ium bromide (Compound No. 72)

To the solution of the Compound No. 70 (25.0 mg) in acetonitrile (1.5 ml), benzylbromide (excess) was added and the reaction mixture was stirred at 55° C. overnight and subsequently at room temperature for further 24 hours. The reaction mixture was then concentrated under reduced pressure. The residue was washed with diethyl ether several times and dried under vacuum to furnish the desired compound. Yield: 34 mg

¹H NMR (CD₃OD) δ: 7.63-7.42 (5H, m), 7.28-7.22 (6H, m), 7.07 (1H, s), 5.46 (2H, s), 4.53-4.51 (4H, m), 3.62-3.68 (1H, m), 2.42-2.45 (1H, m), 1.56-1.49 (9H, m), 1.35-1.32 (9H, m).

Following analogues were prepared similarly,

### 3-Benzyl-1-[2-({(2R)-2-[(1S)-3,3-difluorocyclopentyl]-2-hydroxy-2-phenylacetyl}oxy)ethyl]-2-isopropyl-1H-imidazol-3-ium bromide (Compound No. 73)

¹H NMR (CD₃OD) δ: 7.50-7.41 (5H, m), 7.31-7.28 (4H, m), 7.23-7.22 (2H, m), 7.15-7.14 (1H, m), 5.45 (2H, s), 4.55-4.51 (4H, m), 3.69-3.65 (1H, m), 3.12-3.11 (1H, m), 2.06-2.01 (4H, m), 1.57-1.54 (1H, m), 1.38-1.28 (7H, m).

### 3-Benzyl-1-[2-({(2R)-2-[(1R)-3,3-difluorocyclopentyl]-2-hydroxy-2-phenylacetyl}oxy)ethyl]-2-isopropyl-1H-imidazol-3-ium bromide (Compound No. 74)

¹H NMR (CD₃OD) δ: 7.49-7.42 (5H, m), 7.30-7.27 (6H, m), 7.12-7.11 (1H, m), 5.45 (2H, s), 4.56-4.51 (4H, m), 3.67-3.65 (1H, m), 3.13-3.12 (1H, m), 2.06-1.30 (12H, m).

### 3-(4-Bromobenzyl)-1-[2-({(2R)-2-[(1R)-3,3-difluorocyclopentyl]-2-hydroxy-2-phenylacetyl}oxy)ethyl]-2-isopropyl-1H-imidazol-3-ium bromide (Compound No. 75)

¹H NMR (CD₃OD) δ: 7.61-7.59 (1H, m), 7.50-7.47 (2H, m), 7.33-7.28 (5H, m), 7.15-7.10 (3H, m), 5.43 (2H, s), 4.57-4.50 (4H, m), 3.67-3.63 (1H, m), 3.19-3.18 (1H, m), 2.16-1.76 (6H, m), 1.34-1.30 (6H, m).

### 3-Benzyl-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 76)

¹H NMR (CD₃OD) δ: 7.46-7.25 (12H, m), 5.30 (2H, s), 4.54-4.49 (2H, m), 4.43-4.41 (2H, m), 3.13-3.12 (1H, m), 2.48 (3H, s), 1.78-1.74 (6H, m).

### 3-(4-Bromobenzyl)-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 77)

¹H NMR (CD₃OD) δ: 7.61-7.59 (2H, m), 7.48-7.45 (3H, m), 7.32-7.30 (4H, m), 7.21-7.18 (2H, m), 5.30 (2H, s), 4.55-4.43 (4H, m), 3.13-3.11 (1H, m), 2.47 (3H, s), 2.18-1.95 (2H, m), 1.81-1.73 (4H, m).

### 3-(4-Fluorobenzyl-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 78)

¹H NMR (CD₃OD) δ: 7.48-7.46 (2H, m), 7.38-7.31 (6H, m), 7.25-7.24 (1H, m), 7.20-7.15 (2H, m), 5.29 (2H, m), 4.55-4.43 (4H, m), 3.15-3.11 (1H, m), 2.49 (3H, s), 2.18-1.73 (6H, m).

### 3-Benzyl-1-(2-{[cycloheptyl(hydroxy)phenylacetyl]oxy}ethyl)-2-methyl-1H-imidazol-3-ium bromide (Compound No. 79).

¹H NMR (CD₃OD) δ: 7.20-7.66 (m, 12H), 5.31 (m, 2H), 4.41-4.47 (m, 4H), 2.42-2.47 (m, 4H), 1.21-1.75 (m, 12H).

### 3-(4-Bromobenzyl)-1-(2-{[cycloheptyl(hydroxy)phenylacetyl]oxy}ethyl)-2-methyl-1H-imidazol-3-ium bromide (Compound No. 80)

¹H NMR (CD₃OD) δ: 7.18-7.68 (m, 11H), 5.28-5.29 (m, 2H), 4.41-4.55 (m, 4H), 2.40-2.47 (m, 4H), 1.19-1.59 (m, 12H).

### 1-[2-({(2R)-2-[(1R)-3,3-Difluorocyclopentyl]-2-hydroxy-2-phenylacetyl}oxy)ethyl]-3-(4-fluorobenzyl)-2-isopropyl-1H-imidazol-3-ium bromide (Compound No. 82)

¹H NMR (CD₃OD) δ: 7.10-7.67 (m, 11H), 5.43 (s, 2H), 4.47-4.57 (m, 4H), 3.66-3.69 (q, 1H), 3.19 (m, 1H), 1.28-2.10 (m, 12H).

### 3-Benzyl-1-[3-({(2R)-2-[(1S)-3,3-difluorocyclopentyl]-2-hydroxy-2-phenylacetyl}oxy)propyl]-2-methyl-1H-imidazol-3-ium bromide (Compound No. 101)

¹H NMR (CD₃OD) δ: 7.60-7.62 (m, 2H), 7.27-7.48 (m, 10H), 5.34 (s, 2H), 4.12-4.16 (m, 2H), 4.05-4.10 (m, 2H), 3.20 (m, 1H), 2.42 (s, 3H), 1.26-2.15 (m, 8H).

### 3-(4-Bromobenzyl)-1-[3-({(2R)-2-[(1S)-3,3-difluorocyclopentyl]-2-hydroxy-2-phenylacetyl}oxy)propyl]-2-methyl-1H-imidazol-3-ium bromide (Compound No. 102)

¹H NMR (CD₃OD) δ: 7.58-7.63 (m, 4H), 7.20-7.49 (m, 7H), 5.32 (s, 2H), 4.07-4.16 (m, 4H), 3.27-3.28 (m, 1H), 2.42 (s, 3H), 2.12-2.16 (p, 2H), 1.17-2.00 (m, 6H).

### 3-Benzyl-1-[3-({(2R)-2-[(1R)-3,3-difluorocyclopentyl]-2-hydroxy-2-phenylacetyl}oxy)propyl]-2-methyl-1H-imidazol-3-ium bromide (Compound No. 104).

¹H NMR (CD₃OD) δ: 7.60-7.62 (m, 2H), 7.27-7.48 (m, 10H), 5.34 (s, 2H), 4.13-4.17 (m, 2H), 4.05-4.08 (m, 2H), 3.27 (m, 1H), 2.42 (s, 3H), 1.5-2.16 (m, 8H).

### 3-(4-Bromobenzyl)-1-[3-({(2R)-2-[(1R)-3,3-difluorocyclopentyl]-2-hydroxy-2-phenylacetyl}oxy)propyl]-2-methyl-1H-imidazol-3-ium bromide (Compound No. 105)

¹H NMR (CD₃OD) δ: 7.58-7.62 (m, 4H), 7.20-7.50 (m, 7H), 5.33 (s, 2H), 4.13-4.17 (m, 2H), 4.05-4.10 (m, 2H), 3.26 (m, 1H), 2.42 (s, 3H), 1.5-2.16 (m, 8H).

### 3-Benzyl-1-[3-({2R)-2-[(1R or 1S)-3,3-difluorocyclohexyl]-2-hydroxy-2-phenylacetyl}oxy)propyl]-2-methyl-1H-imidazol-3-ium bromide (Compound No. 107)

¹H NMR (CD₃OD) δ: 7.60-7.63 (m, 2H), 7.26-7.48 (m, 10H), 5.34 (s, 2H), 4.13-4.18 (m, 2H), 4.06-4.07 (m, 2H), 2.6 (m, 1H), 2.41 (s, 3H), 2.13-2.16 (p, 2H), 1.28-2.15 (m, 8H).

### 3-4-bromobenzyl-1-[3-({(2R)-2-[(1R or 1S)-3,3-difluorocyclohexyl]-2-hydroxy-2-phenylacetyl}oxy)propyl]-2-methyl-1H-imidazol-3-ium bromide (Compound No. 108)

¹H NMR (CD₃OD) δ: 7.57-7.63 (m, 4H), 7.19-7.49 (m, 7H), 5.32 (s, 2H), 4.13-4.18 (m, 2H), 4.06-4.07 (m, 2H), 2.6 (m, 1H), 2.41 (s, 3H), 2.13-2.16 (p, 2H), 1.28-2.15 (m, 8H).

### Example 7

### Synthesis of 1-(2-{[cyclohexyl(hydroxy)phenyl acetyl]oxy}ethyl)-2,3-dimethyl-1H-imidazol-3-ium iodide (Compound No. 111)

To the solution of Compound No. 60 (43.5 mmoles) in dichloromethane and methanol, methyl iodide (20 equivalent, 875 mmoles) was added and reaction mixture was stirred at room temperature overnight. The reaction mixture was concentrated under reduced pressure. The residue thus obtained was washed with diethyl ether and dried under vacuum to yield the desired compound. Yield: 8 mg.

¹H NMR (CD₃OD) δ: 7.22 (s, 1H), 7.23-7.35 (m, 4H), 7.48-7.51 (m, 2H), 4.38-4.51 (m, 4H), 3.70 (s, 3H), 2.40 (s, 3H), 2.15-2.30 (m, 1H), 1.62-1.84 (m, 3H), 1.14-1.40 (m, 7H).

The following illustrative analogues were prepared similarly,

### 1-[2-({(2R)-2-[(1S)-3,3-difluorocyclopentyl]-2-hydroxy-2-phenylacetyl}oxy)ethyl]-2-isopropyl-3-methyl-1H-imidazol-3-ium iodide (Compound No. 81)

¹H NMR (CD₃OD) δ: 7.48-7.51 (m, 2H), 7.31-7.36 (m, 4H), 7.10 (s, 1H), 4.47-4.87 (m, 4H), 3.85 (s, 3H), 3.61 (q, 1H), 3.10 (q, 1H), 1.27-1.41 (m, 12H).

### 1-(2-{[2-Cyclohexyl-2-hydroxy-2-phenylacetyl]oxy}ethyl)-2-isopropyl-3-methyl-1H-imidazol-3-ium iodide (Compound No. 83)

¹H NMR (CD₃OD) δ: 7.03 (s, 1H), 7.24-7.34 (m, 4H), 7.48-7.50 (m, 2H), 4.46-4.51 (m, 4H), 3.85 (s, 3H), 3.59-3.63 (q, 1H), 2.15-2.25 (m, 1H), 1.62-1.82 (m, 3H), 1.14-1.48 (m, 13H).

### 1-(2-{[Cyclopentyl (hydroxy)phenylacetyl]oxy}ethyl)-3-methyl-1H-imidazol-3-ium iodide (Compound No. 84)

¹H NMR (CD₃OD) δ: 7.29-7.36 (m, 4H), 7.44-7.54 (m, 3H), 4.43-4.54 (m, 4H), 3.80 (s, 3H), 2.92 (q, 1H), 1.27-1.56 (m, 8H).

### 1-[2-({(2R)-2-[(1S)-3,3-difluorocyclopentyl]-2-hydroxy-2-phenylacetyl}oxy)ethyl]-2,3-dimethyl-1H-imidazol-3-ium iodide (Compound No. 85)

¹H NMR (CD₃OD) δ: 7.48-7.51 (m, 2H), 7.26-7.38 (m, 5H), 4.47-4.50 (m, 2H), 4.39-4.42 (m, 2H), 3.70 (s, 3H), 3.08-3.10 (q, 1H), 2.43 (s, 3H), 1.54-2.41 (m, 6H).

### 1-[2-({(2R)-2-[(1R)-3,3-difluorocyclopentyl]-2-hydroxy-2-phenylacetyl}oxy)ethyl]-2,3-dimethyl-1H-imidazol-3-ium iodide (Compound No. 86)

¹H NMR (CD₃OD) δ: 7.33-7.68 (6H, m), 7.23 (1H, s), 4.49-4.52 (m, 2H), 4.38-4.41 (m, 2H), 3.70 (s, 3H), 3.12-3.14 (m, 1H), 3.13 (s, H), 1.76-2.22 (m, 6H).

### 1-(2-{[cyclohexyl(hydroxy)phenylacetyl]oxy}ethyl)-3-methyl-1H-imidazol-3-ium iodide (Compound No. 87)

¹H NMR (CD₃OD) δ: 9.71 (s, 1H), 7.59-7.61 (m, 2H), 7.33-7.42 (m, 3H), 6.93 (s, 1H), 6.65 (s, 1H), 4.52-4.81 (m, 4H), 3.87 (s, 3H), 3.70 (brs, 1H), 2.25 (m, 1H), 1.08-1.77 (m, 10H).

### 1-(2-{[Cyclopentyl(hydroxy)phenylacetyl]oxy}ethyl)-2-isopropyl-3-methyl-1H-imidazol-3-ium iodide (Compound No. 88)

¹H NMR (CD₃OD) δ: 7.49-7.52 (m, 2H), 7.24-7.34 (m, 4H), 7.01 (s, 1H), 4.45-4.49 (m, 4H), 3.85 (s, 3H), 3.58-3.62 (q, 1H), 2.95 (q, 1H), 1.28-1.56 (m, 14H).

### 1-(2-{[Cyclopentyl(hydroxy)phenylacetyl]amino}ethyl)-2,3-dimethyl-1H-imidazol-3-ium iodide (Compound No. 89)

¹H NMR (CD₃OD) δ: 7.55-7.57 (m, 2H), 7.28-7.36 (m, 3H), 7.05-7.09 (m, 2H), 4.09-4.20 (m, 2H), 3.75 (m, 1H), 3.59 (s, 3H), 3.39-3.43 (m, 1H), 3.02-3.05 (m, 1H), 2.31 (s, 3H), 1.17-1.56 (m, 8H).

### 1-(2-{[cyclohexyl(hydroxy)phenylacetyl]amino}ethyl)-2-isopropyl-3-methyl-1H-imidazol-3-ium iodide (Compound No. 90)

¹H NMR (CD₃OD) δ: 7.53-7.55 (m, 2H), 7.27-7.34 (m, 3H), 6.82-6.90 (m, 2H), 4.25-4.87 (m, 2H), 3.77 (s, 3H), 3.30-3.57 (m, 3H), 2.25 (m, 1H), 1.15-1.64 (16H).

### 1-(2-{[cycloheptyl(hydroxy)phenylacetyl]oxy}ethyl)-2,3-dimethyl-1H-imidazol-3-ium iodide (Compound No. 91)

¹H NMR (CD₃OD) δ: 7.50-7.52 (m, 2H), 7.30-7.36 (m, 4H), 7.20 (s, 1H), 4.37-4.51 (m, 4H), 3.71 (m, 3H), 2.41 (m, 4H), 1.20-1.57 (m, 12H).

### 1-(2-{[cycloheptyl(hydroxy)phenylacetyl]oxy}ethyl)-2-isopropyl-3-methyl-1H-imidazol-3-ium iodide (Compound No. 92.)

¹H NMR (CD₃OD) δ: 7.50-7.65 (m, 2H), 7.25-7.35 (m, 4H), 7.02 (s, 1H), 4.47-4.51 (m, 4H), 3.86 (s, 3H), 3.62 (q, 1H), 2.45 (m, 1H), 1.24-1.57 (m, 18H).

### 1-[2-({(2R)-2-[(1R)-3,3-difluorocyclopentyl]-2-hydroxy-2-phenylacetyl}oxy)ethyl]-2-isopropyl-3-methyl-1H-imidazol-3-ium iodide (Compound No. 93)

¹H NMR (CD₃OD) δ: 7.47-7.51 (m, 2H), 7.32-7.37 (m, 3H), 7.25 (s, 1H), 7.06 (s, 1H), 4.47-4.53 (m, 4H), 3.85 (s, 3H), 3.59-3.63 (m, 1H), 3.13-3.15 (m, 1H), 1.38-2.15 (m, 12H).

### 1-(3-{[(2R)-2-(3,3-difluorocyclopentyl)-2-hydroxy-2-phenylacetyl]amino}propyl)-2,3-dimethyl-1H-imidazol-3-ium iodide (Compound No. 94)

¹H NMR (CD₃OD) δ: 7.61-7.65 (m, 2H), 7.27-7.40 (m, 5H), 3.90-3.94 (q, 2H), 3.74 (s, 3H), 3.47-3.48 (m, 2H), 3.16-3.24 (m, 1H), 2.35 (s, 3H), 1.92-1.99 (m, 6H), 1.55 (m, 2H).

### 1-(3-{[Cyclopentyl(hydroxy)phenylacetyl]amino}propyl)-2,3-dimethyl-1H-imidazol-3-ium iodide (Compound No. 95)

¹H NMR (CD₃OD) δ: 7.63-7.66 (m, 2H), 7.24-7.40 (m, 5H), 3.91-3.96 (m, 2H), 3.74 (s, 3H), 3.22-3.24 (m, 2H), 3.14-3.17 (m, 1H), 2.36 (s, 3H), 1.93-1.97 (m, 2H), 1.2-1.63 (8H).

### 1-(3-{[Cyclohexyl(hydroxy)phenylacetyl]amino}propyl)-2,3-dimethyl-1H-imidazol-3-ium iodide (Compound No. 96)

¹H NMR (CD₃OD) δ: 7.62-7.64 (m, 2H), 7.25-7.39 (m, 4H), 7.24-7.25 (m, 1H), 3.88-3.92 (m, 2H), 3.74 (s, 3H), 3.14-3.24 (m, 2H), 2.5 (m, 1H), 2.32 (s, 3H), 1.66-1.68 (m, 2H), 1.02-1.37 (m, 10H).

### 1-(2-{[Cyclopentyl(hydroxy)phenylacetyl](methyl)amino}ethyl)-3-methyl-1H-imidazol-3-ium iodide (Compound No. 97)

¹H NMR (CD₃OD) δ: 8.78 (s, 1H), 7.48-7.54 (d, 2H), 7.33-7.38 (m, 4H), 7.26-7.27 (m, 1H), 4.62 (s, 1H), 4.32 (s, 1H), 3.89 (s, 4H), 3.72-3.78 (m, 1H), 2.87 (s, 3H), 2.78-2.80 (m, 1H), 1.49-1.53 (m, 1H), 3.1-1.38 (m, 3H), 0.99-1.23 (m, 4H)

### 1-(3-{[Cyclopentyl(hydroxy)phenylacetyl](methyl)amino}propyl)-2,3-dimethyl-1H-imidazol-3-ium iodide (Compound No. 98)

¹H NMR (CD₃OD) δ: 7.68 (s, 1H), 7.21-7.52 (m, 6H), 4.90 (bs, 1H), 4.20 (s, 2H), 3.90 (s, 3H), 3.45-3.50 (m, 2H), 3.00 (m, 1H), 2.81-2.83 (m, 6H), 2.15-2.17 (m, 2H), 1.41-1.69 (m, 6H), 1.19-1.22 (m, 2H).

### 1-(3-{[Cyclopentyl(hydroxy)phenylacetyl]oxy}propyl)-2,3-dimethyl-1H-imidazol-3-ium iodide (Compound No. 99)

¹H NMR (CD₃OD) δ: 7.64-7.66 (m, 2H), 7.21-7.41 (m, 5H), 4.11-4.16 (m, 2H), 4.03-4.04 (m, 2H), 3.76 (s, 3H), 3.0-3.1 (m, 1H), 2.41 (s, 3H), 2.09-2.12 (q, 2H), 1.22-1.66 (m, 8H).

### 1-[3-({(2R)-2-[(1S)-3,3-difluorocyclopentyl]-2-hydroxy-2-phenylacetyl}oxy)propyl]-2,3-dimethyl-1H-imidazol-3-ium iodide (Compound No. 100)

¹H NMR (CD₃OD) δ: 7.62-7.64 (m, 2H), 7.23-7.41 (m, 5H), 4.14-4.18 (m, 2H), 4.03-4.08 (m, 2H), 3.76 (s, 3H), 3.22-3.29 (m, 1H), 2.43 (s, 3H), 1.5-2.15 (m, 8H).

### 1-[3-({(2R)-2-[(1R)-3,3-difluorocyclopentyl]-2-hydroxy-2-phenylacetyl}oxy)propyl]-2,3-dimethyl-1H-imidazol-3-ium iodide (Compound No. 103)

¹H NMR (CD₃OD) δ: 7.61-7.63 (m, 2H), 7.23-7.41 (m, 5H), 4.14-4.18 (m, 2H), 4.03-4.08 (m, 2H), 3.76 (s, 3H), 3.22 (m, 1H), 2.41 (s, 3H), 1.5-2.15 (m, 2H).

### 1-[3-({(2R)-2-[(1S or 1R)-3,3-difluorocyclohexyl]-2-hydroxy-2-phenylacetyl}oxy)propyl]-2,3-dimethyl-1H-imidazol-3-ium bromide (Compound No. 106)

¹H NMR (CD₃OD) δ: 7.63-7.64 (m, 2H), 7.22-7.61 (m, 5H), 4.14-4.21 (m, 2H), 4.01-4.07 (m, 2H), 3.75 (s, 3H), 2.6 (m, 1H), 2.41 (s, 3H), 2.10-2.13 (p, 2H), 1.16-2.15 (m, 6H).

### 1-(2-{[Cyclopentyl(hydroxy)phenylacetyl](methyl)amino}ethyl)-2,3-dimethyl-1H-imidazol-3-ium iodide (Compound No. 109)

¹H NMR (CD₃OD) δ: 7.51-7.52 (m, 1AR-H), 7.32-7.35 (m, 5H), 7.18 (s, 1H), 4.42 (m, 2H), 4.16 (m, 1H), 3.98 (s, 3H), 3.65-3.66 (m, 1H), 2.96 (s, 4H), 2.83 (s, 3H), 1.19-1.67 (m, 8H).

### Biological Activity

### Radioligand Binding Assays:

The affinity of test compounds for M₁, M₂ and M₃ muscarinic receptor subtypes was determined by [³H]-N-methylscopolamine binding studies using rat heart and submandibular gland respectively as described by Moriya et al., (Life Sci, (1999), 64(25): 2351-2358) with minor modifications. In competition binding studies, specific binding of [³H]NMS was also determined using membranes from Chinese hamster ovary (CHO) cells expressing cloned human M₁, M₂, M₃, M₄ and M₅ receptors. Selectivities were calculated from the Ic values obtained on these human cloned membranes.

Membrane preparation: Submandibular glands and heart were isolated and placed in ice-cold homogenizing buffer (HEPES 20 mM, 10 mM EDTA, pH 7.4) immediately after sacrifice. The tissues were homogenized in 10 volumes of homogenizing buffer and the homogenate was filtered through two layers of wet gauze and filtrate was centrifuged at 500 g for 10 minutes at 4° C. The supernatant was subsequently centrifuged at 40,000 g for 20 min. at 4° C. The pellet thus obtained was resuspended in assay buffer (HEPES 20 mM, EDTA 5 mM, pH 7.4) and were stored at −70° C. until the time of assay.

Ligand binding assay: The compounds were dissolved and diluted in DMSO. The membrane homogenates (150-250 μg protein) were incubated in 250 μl of assay volume (HEPES 20 mM, pH 7.4) at 24-25° C. for 3 hours Non-specific binding was determined in the presence of 1 μM atropine. The incubation was terminated by vacuum filtration over GF/B fiber filters (Wallac). The filters were then washed with ice-cold 50 mM Tris HCl buffer (pH 7.4). The filter mats were dried and bound radioactivity retained on filters was counted. The IC₅₀ & K_{d} were estimated by using the non-linear curve fitting program using G Pad Prism software. The value of inhibition constant Kᵢ was calculated from competitive binding studies by using Cheng & Prusoff equation (Biochem. Pharmacol., (1973), 22: 3099-3108), Kᵢ=IC₅₀/(1+L/K_{d}), where L is the concentration of [³H]NMS used in the particular experiment. pKᵢ is −log [Kᵢ].

Compounds described herein showed activity towards M3 receptors in the range of from about 1000 nM to about 0.02 nM, for example from about 100 nM to about 0.02 nM, or for example, from about 50 nM to about 0.02 nM, or for example, from about 10 nM to about 0.02 nM, or for example, from about 1 nM to about 0.02 nM.

Particular compounds described herein (compound Nos. 2, 8, 9, 13-16, 18, 20, 22-25, 27-31, 33-38, 46-60, 64-69, 72-109 and 111) also showed activity towards M2 receptors in the range of from about 1000 nM to about 0.3 nM, or for example, from about 500 nM to about 0.3 nM, or for example, from about 100 nM to about 0.3 nM, or for example, from about 50 nM to about 0.3 nM, or for example, from about 10 nM to about 0.3 nM, or for example, from about 1 nM to about 0.3 nM.

The ratio of M2/M3 activities (the division of the M2 activity value by the M3 activity value) for tested compounds (compound Nos. 2, 8, 9, 13-16, 18, 20, 22-25, 27-31, 33-38, 46-60, 64-69, 72-109 and 111) ranged from about 2 to about 128, or for example, from about 10 to about 128, or for example, from about 25 to about 128.

### Functional Experiments Using Isolated Rat Bladder:

### Methodology:

Animals are euthanized by overdose of thiopentone and whole bladder is isolated and removed rapidly and placed in ice cold Tyrode buffer with the following composition (mMol/L) NaCl 137; KCl 2.7; CaCl₂ 1.8; MgCl₂ 0.1; NaHCO₃ 11.9; NaH₂PO₄ 0.4; Glucose 5.55 and continuously gassed with 95% O₂ and 5% CO₂.

The bladder is cut into longitudinal strips (3 mm wide and 5-6 mm long) and mounted in 10 ml organ baths at 30° C., with one end connected to the base of the tissue holder and the other end connected through a force displacement transducer. Each tissue is maintained at a constant basal tension of 1 g and allowed to equilibrate for 1^{1/2} hour during which the Tyrode buffer is changed every 15-20 minutes. At the end of equilibration period the stabilization of the tissue contractile response is assessed with 1 μmol/L of carbachol till a reproducible response is obtained. Subsequently a cumulative concentration response curve to carbachol (10⁻⁹ mol/L to 3×10⁻⁴ mol/L) was obtained. After several washes, once the baseline is achieved, cumulative concentration response curve is obtained in presence of NCE (NCE added 20 minutes prior to the second cumulative response curve.

The contractile results are expressed as % of control E max. ED₅₀ values are calculated by fitting a non-linear regression curve (Graph Pad Prism). pK_{b} values are calculated by the formula pK_{b}=−log [(molar concentration of antagonist/(dose ratio−1))]

where,
dose ratio=ED₅₀ in the presence of antagonist/ED₅₀ in the absence of antagonist.

### In-Vitro Functional Assay

### Animals and Anaesthesia:

Procure Guinea Pig (400-600 g) and remove trachea under anesthesia (sodium pentobarbital, 300 mg/kg i.p) and immediately keep it in ice-cold Krebs Henseleit buffer. Indomethacin (10 uM) is present throughout the KH buffer to prevent the formation of bronchoactive prostanoids.

### Trachea Experiments:

Clean the tissue off adherent fascia and cut it into strips of equal size (with approx. 4-5 tracheal rings in each strip). Remove the epithelium by careful rubbing, minimizing damage to the smooth muscle. Open the trachea along the mid-dorsal surface with the smooth muscle band intact and make a series of transverse cuts from alternate sides so that they do not transect the preparation completely. Tie opposite end of the cut rings with the help of a thread. Mount the tissue in isolated tissue baths containing 10 ml Krebs Henseleit buffer maintained at 37° C. and bubbled with carbogen, at a basal tension of 1 g. Change the buffer 4-5 times for about an hour. Equilibrate the tissue for 1 hr for stabilization. After 1 hour, challenge the tissue with 1 uM carbachol. Repeat this after every 2-3 washes till two similar consecutive responses are obtained. At the end of stabilization, wash the tissues for 30 minutes followed by incubation with suboptimal dose of MRA/Vehicle for 20 minutes prior to contraction of the tissues with 1 μM carbachol. Record the contractile response of tissues either on Powerlab data acquisition system or on Grass polygraph (Model 7). Express the relaxation as percentage of maximum carbachol response. Express the data as mean±s.e. mean for n observations. Calculate the EC₅₀ as the concentration producing 50% of the maximum relaxation to 1 μM carbachol. Compare percent relaxation between the treated and control tissues using non-parametric unpaired t-test. A p value of <0.05 is considered to be statistically significant.

In-Vitro Functional Assay to Evaluate Efficacy of “MRA” in Combination with “PDE-IV Inhibitors”

### Animals and Anaesthesia:

Procure Guinea Pig (400-600 g) and remove trachea under anesthesia (sodium pentobarbital, 300 mg/kg i.p) and immediately keep it in ice-cold Krebs Henseleit buffer. Indomethacin (10 uM) is present throughout the KH buffer to prevent the formation of bronchoactive prostanoids.

Particular compounds described herein (compound Nos. 69, 76-78, 82, 86, 91, 93, 103-105, 107, and 108) showed p_{KB} of from about 7.53±0.08 to about 9.56±0.20.

### Trachea Experiments:

Clean the tissue off adherent fascia and cut it into strips of equal size (with approx. 4-5 tracheal rings in each strip). Remove the epithelium by careful rubbing, minimizing damage to the smooth muscle. Open the trachea along the mid-dorsal surface with the smooth muscle band intact and make a series of transverse cuts from alternate sides so that they do not transect the preparation completely. Tie opposite end of the cut rings with the help of a thread. Mount the tissue in isolated tissue baths containing 10 ml Krebs Henseleit buffer maintained at 37° C. and bubbled with carbogen, at a basal tension of 1 g. Change the buffer 4-5 times for about an hour. Equilibrate the tissue for 1 hour for stabilization. After 1 hour, challenge the tissue with 1 uM carbachol. Repeat this after every 2-3 washes till two similar consecutive responses are obtained. At the end of stabilization, wash the tissues for 30 minutes followed by incubation with suboptimal dose of MRA/Vehicle for 20 minutes prior to contraction of the tissues with 1 μM carbachol and subsequently assess the relaxant activity of the PDE-IV inhibitor [10⁻⁹ M to 10⁻⁴ M] on the stabilized developed tension/response. Record the contractile response of tissues either on Powerlab data acquisition system or on Grass polygraph (Model 7). Express the relaxation as percentage of maximum carbachol response. Express the data as mean±s.e. mean for n observations. Calculate the EC₅₀ as the concentration producing 50% of the maximum relaxation to 1 μM carbachol. Compare percent relaxation between the treated and control tissues using non-parametric unpaired t-test. A p value of <0.05 is considered to be statistically significant.

### In-Vivo Assay to Evaluate Efficacy of MRA Inhibitors

Male Guinea pig were anesthetized with urethane (1.5 g/kg, i.p.). Trachea was cannulated along with jugular vein (for carbachol challenge) and animals were placed in the Plethysmograph-Box (PLY 3114 model; Buxco Electronics, Sharon, USA.). Respiratory parameters were recorded using Pulmonary Mechanics Analyzer, Biosystems XA software (Buxco Electronics, USA), which calculated lung resistance (R_{L}) on a breath-by-breath basis. Bronchoconstriction was induced by injections of Carbachol (10 μg/kg) delivered into the jugular vein. Increase in R_{L} over a period of 5 minutes post carbachol challenge was recorded in presence or absence of MRA or vehicle at 2 hours and 12 hours post treatment and expressed as % increase in R_{L} from basal.

In-Vivo Assay to Evaluate Efficacy of MRA in Combination with PDE-IV Inhibitors

### Drug Treatment:

MRA (1 μg/kg to 1 mg/kg) and PDE-IV inhibitor (1 μg/kg to 1 mg/kg) are instilled intratracheally under anesthesia either alone or in combination.

### Method:

Male wistar rats weighing 200±20 g are used in the study. Rats have free access to food and water. On the day of experiment, animals are exposed to lipopolysaccharide (LPS, 100 μg/ml) for 40 minutes One group of vehicle treated rats is exposed to phosphate buffered saline (PBS) for 40 minutes Two hours after LPS/PBS exposure, animals are placed inside a whole body plethysmograph (Buxco Electronics, USA) and exposed to PBS or increasing acetylcholine (1, 6, 12, 24, 48 and 96 mg/ml) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchoalveolar lavage (BAL) is performed. Collected lavage fluid is centrifuged at 3000 rpm for 5 minutes at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells ml⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS

The percent inhibition data is used to compute ED₅₀ vales using Graph Pad Prism software (Graphpad Software Inc., USA).

In-Vivo Assay to Evaluate Efficacy of MRA in Combination with Corticosteroids

### Ovalbumin Induced Airway Inflammation:

Guinea pigs are sensitised on days 0, 7 and 14 with 50-μg ovalbumin and 10 mg aluminium hydroxide injected intraperitoneally. On days 19 and 20 guinea pigs are exposed to 0.1% w v⁻¹ ovalbumin or PBS for 10 minutes and with 1% ovalbumin for 30 minutes on day 21. Guinea pigs are treated with test compound (0.1, 0.3 and 1 mg kg⁻¹) or standard 1 mg kg⁻¹ or vehicle once daily from day 19 and continued for 4 days. Ovalbumin/PBS challenge is performed 2 hours after different drug treatment.

24 hours after the final ovalbumin challenge BAL is performed using Hank's balanced salt solution (HBSS). Collected lavage fluid is centrifuged at 3000 rpm for 5 minutes at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and eosinophil count are expressed as cell count (millions cells ml⁻¹ of BAL). Eosinophil is also expressed as percent of total leukocyte count. % inhibition is computed using the following formula.

$$\% Inhibition = \frac{{Eos}_{OVA} - {Eos}_{TEST}}{{Eos}_{OVA} - {Eos}_{CON}} \times 100$$

Where,

Eos_{OVA}=Percentage of eosinophil in untreated ovalbumin challenged group
Eos_{TEST}=Percentage of eosinophil in group treated with a given dose of test compound
Eos_{CON}=Percentage of eosinophil in group not challenged with ovalbumin.
In-Vivo Assay to Evaluate Efficacy of “MRA” in Combination with P38 Map Kinase Inhibitors

Lipopolysaccharide (LPS) induced airway hyperreactivity (AHR) and neutrophilia:

### Drug Treatment:

MRA (1 μg/kg to 1 mg/kg) and p38 MAP kinase inhibitor (1 μg/kg to 1 mg/kg) are instilled intratracheally under anesthesia either alone or in combination.

### Method:

Male wistar rats weighing 200±20 gM are used in the study. Rats have free access to food and water. On the day of experiment, animals are exposed to lipopolysaccharide (LPS, 100 μg/ml) for 40 minutes One group of vehicle treated rats is exposed to phosphate buffered saline (PBS) for 40 minutes Two hours after LPS/PBS exposure, animals are placed inside a whole body plethysmograph (Buxco Electronics, USA) and exposed to PBS or increasing acetylcholine (1, 6, 12, 24, 48 and 96 mg/ml) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchoalveolar lavage (BAL) is performed. Collected lavage fluid is centrifuged at 3000 rpm for 5 minutes, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells ml⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS

The percent inhibition data is used to compute ED₅₀ vales using Graph Pad Prism software (Graphpad Software Inc., USA).

In-Vivo Assay to Evaluate Efficacy of “MRA” in Combination with β2-Agonists

### Drug Treatment:

MRA (1 μg/kg to 1 mg/kg) and long-acting β₂ agonist are instilled intratracheally under anesthesia either alone or in combination.

### Method

Wistar rats (250-350 gm) or balb/C mice (20-30 gM) are placed in body box of a whole body plethysmograph (Buxco Electronics., USA) to induce bronchoconstriction. Animals are allowed to acclimatise in the body box and are given successive challenges, each of 2 min duration, with PBS (vehicle for acetylcholine) or acetylcholine (i.e. 24, 48, 96, 144, 384, and 768 mg/ml). The respiratory parameters are recorded online using Biosystem XA software, (Buxco Electronics, USA) for 3 minutes A gap of 2 minutes is allowed for the animals to recover and then challenged with the next higher dose of acetylcholine (ACh). This step is repeated until Penh of rats attained 2 times the value (PC-100) seen with PBS challenge. Following PBS/ACh challenge, Penh values (index of airway resistance) in each rat/mice is obtained in the presence of PBS and different doses of ACh. Penh, at any chosen dose of ACh is, expressed as percent of PBS response. The Penh values thus calculated are fed into Graph Pad Prism software (Graphpad Software Inc., USA) and using a nonlinear regression analysis PC100 (2 folds of PBS value) values are computed. % inhibition is computed using the following formula.

$$\% Inhibition = \frac{PC {100}_{TEST} - PC {100}_{CON}}{768 - PC {100}_{CON}} \times 100$$

Where,

PC100_{CON}=PC100 in vehicle treated group
PC100_{TEST}=PC100 in group treated with a given dose of test compound
768=is the maximum amount of acetylcholine used.

While the present invention has been described in terms of its specific embodiments, certain modification and equivalents will be apparent to those skilled in the art and are intended.

We claim:

1. Compounds having the structure of Formula I: 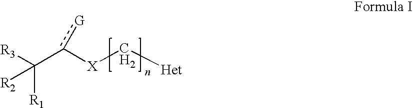
and its pharmaceutically accepted salts, pharmaceutically acceptable solvates, enantiomers, diastereomers, polymorphs or N-oxides wherein [symbol]
represents a single bond when G is —OH and double bond when G is —O;
R₁ and R₂ are independently selected from hydrogen, alkyl, alkenyl, alkynyl, aralkyl, cycloalkyl, aryl, heteroaryl, heterocyclyl, heterocyclylalkyl or heteroarylalkyl;
R₃ is selected from the group selected from hydrogen, hydroxy, alkoxy, alkenyloxy or alkynyloxy;
X is selected from oxygen, —NH, —NR (wherein R is alkyl, alkenyl, alkenyl, alkynyl or aryl), sulphur or no atom;
Het is heterocyclyl or heteroaryl; and
n is an integer from 1 to 6,
with the proviso that when R₁ and R₂ are phenyl, R₃ is hydroxy and X is no atom, then Het cannot be a saturated heterocyclyl group.

2. A compound selected from the group consisting of
2-Cyclopentyl-2-hydroxy-N-[2-(1H-imidazol-1-yl)ethyl]-2-phenylacetamide (Compound No. 1);
1H-Imidazol-1-ylmethyl cyclohexyl(hydroxy)(4-methylphenyl)acetate (Compound No. 2)
2-(4-Fluorophenyl)-2-hydroxy-N-[2-(1H-imidazol-1-yl)ethyl]-2-phenylacetamide (Compound No. 3);
2-Cyclobutyl-2-hydroxy-N-[2-(1H-imidazol-1-yl)ethyl]-2-phenylacetamide (Compound No. 4);
2-Cyclopentyl-2-(4-fluorophenyl)-2-hydroxy-N-[2-(1H-imidazol-1-yl)ethyl]acetamide (Compound No. 5);
2-Hydroxy-N-[2-(1H-imidazol-1-yl)ethyl]-2,2-diphenylacetamide (Compound No. 6);
2-Cyclohexyl-2-hydroxy-N-[2-(1H-imidazol-1-yl)ethyl]-2-phenylacetamide (Compound No. 7);
2-Cyclopentyl-2-hydroxy-N-[2-(2-isopropyl-1H-imidazol-1-yl)ethyl]-2-phenylacetamide (Compound No. 8);
2-Cyclohexyl-2-hydroxy-N-[2-(2-isopropyl-1H-imidazol-1-yl)ethyl]-2-phenylacetamide (Compound No. 9);
2-Hydroxy-N-[2-(2-isopropyl-1H-imidazol-1-yl)ethyl]-2-phenyl-2-pyridin-3-ylacetamide (Compound No. 10);
2-(4-Fluorophenyl)-2-hydroxy-N-[2-(2-isopropyl-1H-imidazol-1-yl)ethyl]-2-phenylacetamide (Compound No. 11);
2-Hydroxy-N-[2-(2-isopropyl-1H-imidazol-1-yl)ethyl]-2,2-diphenylacetamide (Compound No. 12);
2-(2-Methyl-1H-imidazol-1-yl)ethyl cyclopentyl(hydroxy)phenylacetate (Compound No. 13);
2-(2-Methyl-1H-imidazol-1-yl)ethyl (2R)-cyclopentyl(hydroxy)phenylacetate (Compound No. 14);
1-Cyclopentyl-1-hydroxy-1-(4-methoxyphenyl)-3-(2-methyl-1H-imidazol-1-yl)acetone (Compound No. 15);
(2R)-2-cyclopentyl-2-hydroxy-N-[2-(2-methyl-1H-imidazol-1-yl)ethyl]-2-phenylacetamide (Compound No. 16);
2-Hydroxy-N-[2-(1H-imidazol-1-yl)ethyl]-2-phenyl-2-pyridin-3-ylacetamide (Compound No. 17);
2-Cyclopentyl-2-hydroxy-N-[2-(2-methyl-1H-imidazol-1-yl)ethyl]-2-phenylacetamide (Compound No. 18);
2-Hydroxy-N-[2-(2-methyl-1H-imidazol-1-yl)ethyl]-2-phenyl-2-pyridin-3-ylacetamide (Compound No. 19);
2-Cyclohexyl-2-hydroxy-N-[2-(2-methyl-1H-imidazol-1-yl)ethyl]-2-phenylacetamide (Compound No. 20);
2-(4-Fluorophenyl)-2-hydroxy-N-[2-(2-methyl-1H-imidazol-1-yl)ethyl]-2-phenylacetamide (Compound No. 21);
2-Cyclopentyl-2-(4-fluorophenyl)-2-hydroxy-N-[2-(2-methyl-1H-imidazol-1-yl)ethyl]acetamide (Compound No. 22);
2-Cyclobutyl-2-hydroxy-N-[2-(2-methyl-1H-imidazol-1-yl)ethyl]-2-phenylacetamide (Compound No. 23);
2-Hydroxy-N-[2-(2-methyl-1H-imidazol-1-yl)ethyl]-2,2-diphenylacetamide (Compound No. 24);
3,3,3-Trifluoro-2-hydroxy-N-[2-(2-methyl-1H-imidazol-1-yl)ethyl]-2-(4-methylphenyl)propanamide (Compound No. 25);
N-[2-(2-methyl-1H-imidazol-1-yl)ethyl]-2,2-diphenylacetamide (Compound No. 26);
2-Cyclopentyl-N-[2-(2-methyl-1H-imidazol-1-yl)ethyl]-2-phenylacetamide (Compound No. 27);
2-Cyclopentyl-2-hydroxy-N-[2-(2-methyl-1H-imidazol-1-yl)ethyl]-2-(4-methylphenyl)acetamide (Compound No. 28);
2-Cyclohexyl-2-hydroxy-N-[2-(2-methyl-1H-imidazol-1-yl)ethyl]-2-(4-methylphenyl)acetamide (Compound No. 29);
2-Hydroxy-N-[2-(2-methyl-1H-imidazol-1-yl)ethyl]-2-(4-methylphenyl)-2-phenylacetamide (Compound No. 30);
2-Cyclopentyl-2-hydroxy-N-[3-(1H-imidazol-1-yl)propyl]-2-phenylacetamide (Compound No. 31);
2-Cyclohexyl-2-hydroxy-N-[3-(1H-imidazol-1-yl)propyl]-2-phenylacetamide (Compound No. 32);
2-Cyclopentyl-2-hydroxy-N-[3-(2-methyl-1H-imidazol-1-yl)propyl]-2-phenylacetamide (Compound No. 33);
2-Cyclohexyl-2-hydroxy-N-[3-(2-methyl-1H-imidazol-1-yl)propyl]-2-phenylacetamide (Compound No. 34);
(2R)-2-(3,3-Difluorocyclopentyl)-2-hydroxy-N-[3-(2-methyl-1H-imidazol-1-yl)propyl]-2-phenylacetamide (Compound No. 35);
2-Cyclopentyl-2-hydroxy-N-methyl-N-[3-(2-methyl-1H-imidazol-1-yl)propyl]-2-phenylacetamide (Compound No. 36);
2-Cyclohexyl-2-hydroxy-N-methyl-N-[3-(2-methyl-1H-imidazol-1-yl)propyl]-2-phenylacetamide (Compound No. 37);
2-Cyclopentyl-2-hydroxy-N-[2-(1H-imidazol-1-yl)ethyl]-N-methyl-2-phenylacetamide (Compound No. 38);
2-Cyclopentyl-2-hydroxy-N-[2-(1H-imidazol-4-yl)ethyl]-2-phenylacetamide (Compound No. 39);
N-[2-(1-benzyl-1H-imidazol-4-yl)ethyl]-2-cyclopentyl-2-hydroxy-2-(4-methylphenyl)acetamide (Compound No. 40);
2-Cyclopentyl-2-hydroxy-N-[2-(1H-imidazol-4-yl)ethyl]-2-(4-methylphenyl)acetamide (Compound No. 41);
2-Cyclohexyl-2-hydroxy-N-[2-(1H-imidazol-4-yl)ethyl]-2-(4-methylphenyl)acetamide (Compound No. 42);
2-Hydroxy-N-[2-(1H-imidazol-4-yl)ethyl]-2,2-diphenylacetamide (Compound No. 43);
N-[2-(1-benzyl-1H-imidazol-4-yl)ethyl]-2-cyclohexyl-2-hydroxy-2-(4-methylphenyl)acetamide (Compound No. 44);
N-[2-(1-benzyl-1H-imidazol-4-yl)ethyl]-2-hydroxy-2,2-diphenylacetamide (Compound No. 45);
1H-imidazol-1-ylmethyl cyclopentyl(hydroxy)phenylacetate (Compound No. 46);
1H-imidazol-1-ylmethyl cyclohexyl(hydroxy)phenylacetate (Compound No. 47);
1H-imidazol-1-ylmethyl (2R)-cyclopentyl(hydroxy)phenylacetate (Compound No. 48);
1H-Imidazol-1-ylmethyl cyclopentyl(hydroxy)(4-methoxyphenyl)acetate (Compound No. 49);
1-Cyclohexyl-1-hydroxy-3-(1H-imidazol-1-yl)-1-phenylacetone (Compound No. 50);
1-Cyclohexyl-1-hydroxy-3-(2-methyl-1H-imidazol-1-yl)-1-phenylacetone (Compound No. 51);
1-Cyclopentyl-1-hydroxy-3-(2-isopropyl-1H-imidazol-1-yl)-1-(4-methoxyphenyl)acetone (Compound No. 52);
1-Cyclohexyl-1-hydroxy-3-(2-isopropyl-1H-imidazol-1-yl)-1-phenylacetone (Compound No. 53);
1-Cyclohexyl-1-hydroxy-3-(2-methyl-4,5-dihydro-1H-imidazol-1-yl)-1-phenylacetone (Compound No. 54);
1-Cyclopentyl-1-hydroxy-1-(4-methoxyphenyl)-3-(2-methyl-4,5-dihydro-1H-imidazol-1-yl)acetone (Compound No. 55)
1-Cyclopentyl-1-hydroxy-3-(1H-imidazol-1-yl)-1-(4-methoxyphenyl)acetone (Compound No. 56);
2-Cyclohexyl-2-hydroxy-N-[2-(1H-imidazol-1-yl)ethyl]-N-methyl-2-phenylacetamide (Compound No. 57);
2-Cyclopentyl-2-hydroxy-N-[2-(1H-imidazol-1-yl)ethyl]-N-methyl-2-phenylacetamide (Compound No. 58);
2-(2-Methyl-1H-imidazol-1-yl)ethyl cyclopentyl(phenyl)acetate (Compound No. 59);
2-(2-Methyl-1H-imidazol-1-yl)ethyl cyclohexyl(hydroxy)phenylacetate (Compound No. 60);
3-(2-Methyl-1H-imidazol-1-yl)propyl cyclopentyl(hydroxy)phenylacetate (Compound No. 61);
3-(2-Methyl-1H-imidazol-1-yl)propyl (2R)-[(1R)-3,3-difluorocyclopentyl](hydroxy)phenylacetate (Compound No. 62);
3-(2-Methyl-1H-imidazol-1-yl)propyl 2R-2-(1S or 1R) (3,3-difluoro cyclohexyl)(hydroxy)phenylacetate (Compound No. 63);
2-(2-Isopropyl-1H-imidazol-1-yl)ethyl cyclohexyl(hydroxy)phenylacetate (Compound No. 64);
2-(1H-Imidazol-1-yl)ethyl cyclopentyl(hydroxy)phenylacetate (Compound No. 65);
2-(2-Methyl-1H-imidazol-1-yl)ethyl (2R)-[(1S)-3,3-difluorocyclopentyl](hydroxy)phenylacetate (Compound No. 66);
2-(1H-Imidazol-1-yl)ethyl cyclohexyl(hydroxy)phenylacetate (Compound No. 67);
2-(2-Isopropyl-1H-imidazol-1-yl)ethyl cyclopentyl(hydroxy)phenylacetate (Compound No. 68);
2-(2-Methyl-1H-imidazol-1-yl)ethyl (2R)-[(1R)-3,3-difluorocyclopentyl](hydroxy)phenylacetate (Compound No. 69);
2-(2-Isopropyl-1H-imidazol-1-yl)ethyl cycloheptyl(hydroxy)phenylacetate (Compound No. 70);
2-(2-Methyl-1H-imidazol-1-yl)ethyl cycloheptyl(hydroxy)phenylacetate (Compound No. 71);
3-Benzyl-1-(2-{[cycloheptyl(hydroxy)phenylacetyl]oxy}ethyl)-2-isopropyl-1H-imidazol-3-ium bromide (Compound No. 72);
3-Benzyl-1-[2-({(2R)-2-[(1S)-3,3-difluorocyclopentyl]-2-hydroxy-2-phenylacetyl}oxy)ethyl]-2-isopropyl-1H-imidazol-3-ium bromide (Compound No. 73);
3-Benzyl-1-[2-({(2R)-2-[(1R)-3,3-difluorocyclopentyl]-2-hydroxy-2-phenylacetyl}oxy)ethyl]-2-isopropyl-1H-imidazol-3-ium bromide (Compound No. 74);
3-(4-Bromobenzyl)-1-[2-({(2R)-2-[(1R)-3,3-difluorocyclopentyl]-2-hydroxy-2-phenylacetyl}oxy)ethyl]-2-isopropyl-1H-imidazol-3-ium bromide (Compound No. 75);
3-Benzyl-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 76);
3-(4-Bromobenzyl)-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 77);
3-(4-Fluorobenzyl-1-[2-({(2R)-2-[(1R)-3,3-difluorocyclopentyl]-2-hydroxy-2-phenylacetyl}oxy)ethyl]-2-methyl-1H-imidazol-3-ium bromide (Compound No. 78);
3-Benzyl-1-(2-{[cycloheptyl(hydroxy)phenylacetyl]oxy}ethyl)-2-methyl-1H-imidazol-3-ium bromide (Compound No. 79);
3-(4-Bromobenzyl)-1-(2-{[cycloheptyl(hydroxy)phenylacetyl]oxy}ethyl)-2-methyl-1H-imidazol-3-ium bromide (Compound No. 80);
1-[2-({(2R)-2-[(1S)-3,3-difluorocyclopentyl]-2-hydroxy-2-phenylacetyl}oxy)ethyl]-2-isopropyl-3-methyl-1H-imidazol-3-ium iodide (Compound No. 81);
1-[2-({(2R)-2-[(1R)-3,3-difluorocyclopentyl]-2-hydroxy-2-phenylacetyl}oxy)ethyl]-3-(4-fluorobenzyl)-2-isopropyl-1H-imidazol-3-ium bromide (Compound No. 82);
1-(2-{[2-Cyclohexyl-2-hydroxy-2-phenylacetyl]oxy}ethyl)-2-isopropyl-3-methyl-1H-imidazol-3-ium iodide (Compound No. 83);
1-(2-{[Cyclopentyl (hydroxy)phenylacetyl]oxy}ethyl)-3-methyl-1H-imidazol-3-ium iodide (Compound No. 84);
1-[2-({(2R)-2-[(1S)-3,3-difluorocyclopentyl]-2-hydroxy-2-phenylacetyl}oxy)ethyl]-2,3-dimethyl-1H-imidazol-3-ium iodide (Compound No. 85);
1-[2-({(2R)-2-[(1R)-3,3-difluorocyclopentyl]-2-hydroxy-2-phenylacetyl}oxy)ethyl]-2, 3-dimethyl-1H-imidazol-3-ium iodide (Compound No. 86);
1-(2-{[Cyclohexyl(hydroxy)phenylacetyl]oxy}ethyl)-3-methyl-1H-imidazol-3-ium iodide (Compound No. 87);
1-(2-{[Cyclopentyl(hydroxy)phenylacetyl]oxy}ethyl)-2-isopropyl-3-methyl-1H-imidazol-3-ium iodide (Compound No. 88);
1-(2-{[Cyclopentyl(hydroxy)phenylacetyl]amino}ethyl)-2,3-dimethyl-1H-imidazol-3-ium iodide (Compound No. 89);
1-(2-{[Cyclohexyl(hydroxy)phenylacetyl]amino}ethyl)-2-isopropyl-3-methyl-1H-imidazol-3-ium iodide (Compound No. 90);
1-(2-{[Cycloheptyl(hydroxy)phenylacetyl]oxy}ethyl)-2,3-dimethyl-1H-imidazol-3-ium iodide (Compound No. 91);
1-(2-{[Cycloheptyl(hydroxy)phenylacetyl]oxy}ethyl)-2-isopropyl-3-methyl-1H-imidazol-3-ium iodide (Compound No. 92.);
1-[2-({(2R)-2-[(1R)-3,3-difluorocyclopentyl]-2-hydroxy-2-phenylacetyl}oxy)ethyl]-2-isopropyl-3-methyl-1H-imidazol-3-ium iodide (Compound No. 93);
1-(3-{[(2R)-2-(3,3-difluorocyclopentyl)-2-hydroxy-2-phenylacetyl]amino}propyl)-2,3-dimethyl-1H-imidazol-3-ium iodide (Compound No. 94);
1-(3-{[Cyclopentyl(hydroxy)phenylacetyl]amino}propyl)-2,3-dimethyl-1H-imidazol-3-ium iodide (Compound No. 95);
1-(3-{[Cyclohexyl(hydroxy)phenylacetyl]amino}propyl)-2,3-dimethyl-1H-imidazol-3-ium iodide (Compound No. 96);
1-(2-{[Cyclopentyl(hydroxy)phenylacetyl](methyl)amino}ethyl)-3-methyl-1H-imidazol-3-ium iodide (Compound No. 97);
1-(3-{[Cyclopentyl(hydroxy)phenylacetyl](methyl)amino}propyl)-2,3-dimethyl-1H-imidazol-3-ium iodide (Compound No. 98);
1-(3-{[Cyclopentyl(hydroxy)phenylacetyl]oxy}propyl)-2,3-dimethyl-1H-imidazol-3-ium iodide (Compound No. 99);
1-[3-({(2R)-2-[(1S)-3,3-Difluorocyclopentyl]-2-hydroxy-2-phenylacetyl}oxy)propyl]-2, 3-dimethyl-1H-imidazol-3-ium iodide (Compound No. 100);
3-Benzyl-1-[3-({(2R)-2-[(1S)-3,3-difluorocyclopentyl]-2-hydroxy-2-phenylacetyl}oxy)propyl]-2-methyl-1H-imidazol-3-ium bromide (Compound No. 101);
3-(4-Bromobenzyl)-1-[3-({(2R)-2-[(1S)-3,3-difluorocyclopentyl]-2-hydroxy-2-phenylacetyl}oxy)propyl]-2-methyl-1H-imidazol-3-ium bromide (Compound No. 102);
1-[3-({(2R)-2-[(1R)-3,3-difluorocyclopentyl]-2-hydroxy-2-phenylacetyl}oxy)propyl]-2,3-dimethyl-1H-imidazol-3-ium iodide (Compound No. 103);
3-Benzyl-1-[3-({(2R)-2-[(1R)-3,3-difluorocyclopentyl]-2-hydroxy-2-phenylacetyl}oxy)propyl]-2-methyl-1H-imidazol-3-ium bromide (Compound No. 104);
3-(4-Bromobenzyl)-1-[3-({(2R)-2-[(1R)-3,3-difluorocyclopentyl]-2-hydroxy-2-phenylacetyl}oxy)propyl]-2-methyl-1H-imidazol-3-ium bromide (Compound No. 105);
1-[3-({(2R)-2-[(1S or 1R)-3,3-difluorocyclohexyl]-2-hydroxy-2-phenylacetyl}oxy)propyl]-2,3-dimethyl-1H-imidazol-3-ium bromide (Compound No. 106);
3-Benzyl-1-[3-({(2R)-2-[(1R or 1S)-3,3-difluorocyclohexyl]-2-hydroxy-2-phenylacetyl}oxy)propyl]-2-methyl-1H-imidazol-3-ium bromide (Compound No. 107);
3-(4-Bromobenzyl)-1-[3-({(2R)-2-[(1R or 1S)-3,3-difluorocyclohexyl]-2-hydroxy-2-phenylacetyl}oxy)propyl]-2-methyl-1H-imidazol-3-ium bromide (Compound No. 108);
1-(2-{[Cyclopentyl(hydroxy)phenylacetyl](methyl)amino}ethyl)-2,3-dimethyl-1H-imidazol-3-ium iodide (Compound No. 109);
3-(2-Methyl-1H-imidazol-1-yl)propyl (2R)-[(1S)-3,3-difluorocyclopentyl](hydroxy)phenylacetate (Compound No. 110);
1-(2-{[Cyclohexyl(hydroxy)phenyl acetyl]oxy}ethyl)-2,3-dimethyl-1H-imidazol-3-ium iodide (Compound No 111);
2-(2-Isopropyl-1H-imidazol-1-yl)ethyl (2R)-[(1S)-3,3-difluorocyclopentyl](hydroxy)phenylacetate (Compound No. 112); and
2-(2-Isopropyl-1H-imidazol-1-yl)ethyl (2R)-[(1R)-3,3-difluorocyclopentyl](hydroxy)phenylacetate (Compound No. 113).

3. A pharmaceutical composition comprising a therapeutically effective amount of a compound as defined in claim 1 together with pharmaceutically acceptable carriers, excipients or diluents.

4. The use of compounds according to claim 1 for the manufacture of medicament for treating or preventing disorder of the respiratory, urinary and gastrointestinal systems, wherein the disease or disorder is mediated through muscarinic receptors, in animal or human.

5. The use of the compounds as described in claim 4 for the manufacture of medicament for treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

6. The use of the compounds as defined in claim 3 for the manufacture of a medicament for the treating or preventing disease or disorder of the respiratory, urinary and gastroinstestinal systems, wherein the disease or disorder is mediated through muscarinic receptors, in animal or human.

7. The use of the compounds as defined in claim 6 for the manufacture of a medicament for the treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis.

8. A pharmaceutical composition comprising one or more compounds of Formula I 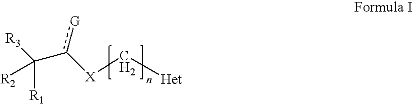
and its pharmaceutically accepted salts, pharmaceutically acceptable solvates, enantiomers, diastereomers, polymorphs or N-oxides wherein [symbol]
represents a single bond when G is —OH and double bond when G is —O;
R₁ and R₂ are independently selected from hydrogen, alkyl, alkenyl, alkynyl, aralkyl, cycloalkyl, aryl, heteroaryl, heterocyclyl, heterocyclylalkyl or heteroarylalkyl;
R₃ is selected from the group selected from hydrogen, hydroxy, alkoxy, alkenyloxy or alkynyloxy;
X is selected from oxygen, —NH, —NR (wherein R is alkyl, alkenyl, alkenyl, alkynyl or aryl), sulphur or no atom;
Het is heterocyclyl or heteroaryl; and
n is an integer from 1 to 6,
with the proviso that when R₁ and R₂ are phenyl, R₃ is hydroxy and X is no atom, then Het cannot be a saturated heterocyclyl group, and
at least one other active ingredient selected from corticosteroids, beta agonists, leukotriene antagonists, 5-lipoxygenase inhibitors, anti-histamines, antitussives, dopamine receptor antagonists, chemokine inhibitors, p38 MAP Kinase inhibitors, and PDE-IV inhibitors.

9. A method of making compounds of Formula IVa, and its pharmaceutically acceptable salts, pharmaceutically acceptable solvates, enantiomers, diastereomers, polymorphs or N-oxides, wherein the method comprises:
a. coupling a compound of Formula II 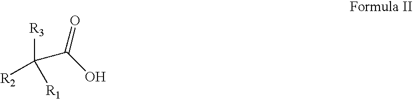
with a compound of formula III 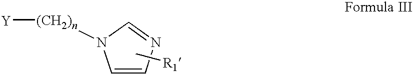
to give a compound of Formula IV; and 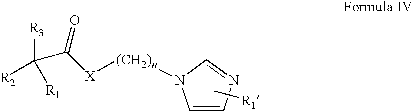
b) reacting a compound of Formula IV with a compound of Formula Q-Z to give a compound of Formula IVa 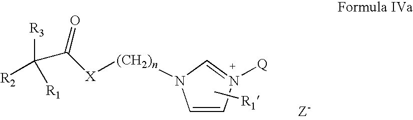
wherein
R₁ and R₂ are independently selected from hydrogen, alkyl, alkenyl, alkynyl, aralkyl, cycloalkyl, aryl, heteroaryl, heterocyclyl, heterocyclylalkyl or heteroarylalkyl;
R₃ is selected from the group selected from hydrogen, hydroxy, alkoxy, alkenyloxy or alkynyloxy;
X is selected from oxygen, —NH, —NR (wherein R is alkyl, alkenyl, alkenyl, alkynyl or aryl), sulphur or no atom;
n is an integer from 1 to 6;
R₁′ is selected from hydrogen, alkyl, alkenyl, alkynyl, aralkyl, cycloalkyl, aryl, heteroaryl, heterocyclyl, heterocyclylalkyl or heteroarylalkyl and R₁′ is always a substitutent on the carbon atoms of imidazolyl ring;
Q is selected from alkyl, alkenyl, alkynyl, cycloalkyl, heterocyclyl, aryl, heteroaryl, aralkyl, heteroarylalkyl or heterocyclylalkyl; and
Z is an anion selected from tartarate, chloride, bromide, iodide, sulphate, phosphate, nitrate, carbonate, fumarate, glutamate, citrate, methanesulphonate, benzenesulphonate, maleate or succinate.

10. A method of making compounds of Formula VIII, and its pharmaceutically acceptable salts, pharmaceutically acceptable solvates, enantiomers, diastereomers, polymorphs or N-oxides, wherein the method comprises:
a. coupling a compound of Formula II 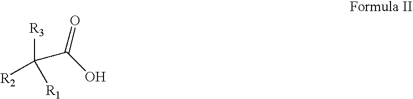
with a compound of Formula V 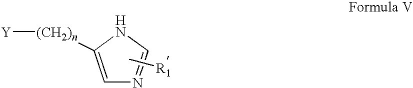
to give a compound of Formula VI; and 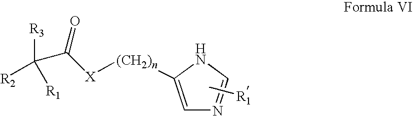
b) N-alkylating a compound of Formula VI with a compound of Formula VII
R₁-hal Formula VII
to give a compound of Formula VIII 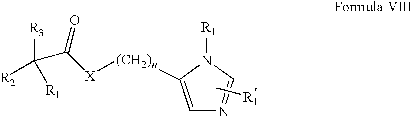
wherein
R₁ and R₂ are independently selected from hydrogen, alkyl, alkenyl, alkynyl, aralkyl, cycloalkyl, aryl, heteroaryl, heterocyclyl, heterocyclylalkyl or heteroarylalkyl;
R₃ is selected from the group selected from hydrogen, hydroxy, alkoxy, alkenyloxy or alkynyloxy;
X is selected from oxygen, —NH, —NR (wherein R is alkyl, alkenyl, alkenyl, alkynyl or aryl), sulphur or no atom;
n is an integer from 1 to 6; and
R₁′ is selected from hydrogen, alkyl, alkenyl, alkynyl, aralkyl, cycloalkyl, aryl, heteroaryl, heterocyclyl, heterocyclylalkyl or heteroarylalkyl and R₁′ is always a substitutent on the carbon atoms of imidazolyl ring.

11. A method of making compounds of Formula XII, and its pharmaceutically acceptable salts, pharmaceutically acceptable solvates, enantiomers, diastereomers, polymorphs or N-oxides, wherein the method comprises:
a. acetylating a compound of Formula II 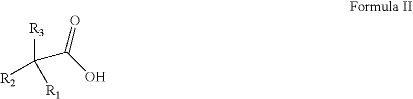
to give a compound of Formula IX; 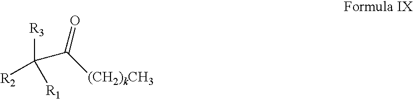
b) halogenating a compound of Formula IX to give a compound of Formula X; and 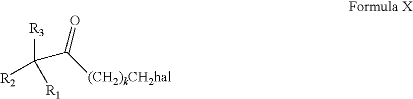
c) coupling of compound of Formula X with compound of Formula XI 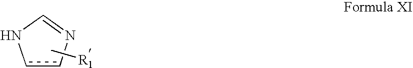
to give a compound of Formula XII, 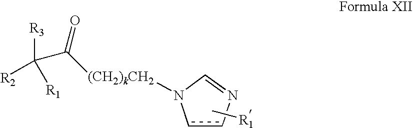
wherein
R₁ and R₂ are independently selected from hydrogen, alkyl, alkenyl, alkynyl, aralkyl, cycloalkyl, aryl, heteroaryl, heterocyclyl, heterocyclylalkyl or heteroarylalkyl;
R₃ is selected from the group selected from hydrogen, hydroxy, alkoxy, alkenyloxy or alkynyloxy;
R₁′ is selected from hydrogen, alkyl, alkenyl, alkynyl, aralkyl, cycloalkyl, aryl, heteroaryl, heterocyclyl, heterocyclylalkyl or heteroarylalkyl and R₁′ is always a substitutent on the carbon atoms of imidazolyl ring;
k is an integer from 0-3; and [symbol]
represents a single bond or double bond.
